(19) United States
(12) Patent Application Publication
(10) Pub. No.: US 2025/0157609 A1
(43) Pub. Date: May 15, 2025

(54) COMPUTER-CONTROLLED SMART REFERRALS SYSTEM AND METHOD

(71) Applicant: Intellectual Frontiers LLC, Silver Spring, MD (US)

(72) Inventor: Shahid N. Shah, Silver Spring, MD (US)

(73) Assignee: Intellectual Frontiers LLC, Silver Spring, MD (US)

(21) Appl. No.: 19/026,240

(22) Filed: Jan. 16, 2025

Related U.S. Application Data
(60) Provisional application No. 63/622,038, filed on Jan. 17, 2024.

(51) Int. Cl. G16H 20/00; G06F 21/60; G06F 21/62; G16H 30/20; G16H 30/40; G16H 50/30

(52) U.S. Cl. CPC G16H 20/00; G06F 21/602; G06F 21/6245; G16H 30/20; G16H 30/40; G16H 50/30

(57) ABSTRACT

An embodiment herein provides a system and method for generating hyper-personalized care pathways. The system includes a data ingestion module configured to receive and process computer executable data from one or more data sources. The system includes a profile generation module operatively coupled to the data ingestion module. The profile generation module is configured to synthesize the computer executable data into a hyper-personalized computer executable profile by applying a machine learning algorithm and predictive analytics. The profile generation module is configured to generate a multi-dimensional computer executable representation of current health status and predicted future healthcare needs of the subject. The system includes a pathway generator module that is configured to determine a set of next best actions for the subject based on the hyper-personalized computer executable profile.

### CROSS-REFERENCE TO RELATED APPLICATION(S)

This application claims the benefit of U.S. Provisional Patent Application No. 63/622,038 filed on Jan. 17, 2024, which is incorporated herein by reference in its entirety.

### BACKGROUND

### Technical Field

The embodiments herein generally relate to digital referral systems and, more particularly, to adaptive computer-controlled systems and methods for synthesizing multi-dimensional subject profiles and acquisition of the profiles in a digital referral system for dynamically determining hyper-personalized care pathways tailored to individual parameters.

### Description of the Related Art

Acquiring of new patients is important for healthcare delivery centers (HDOs) such as hospitals, physician practices, home health organizations, labs, imaging companies, and other ancillary service providers (correctively referred to as HDOs herein). HDOs generally use digital methods to acquire new patients. They leverage digital channels to reach, engage, and retain patients. As shown in FIG. 1, they may use digital methods to acquire new patients. Some of these digital methods may include website and search engine optimization (SEO), Pay-Per-Click (PPC) Advertising, social media, email marketing, telehealth platforms, online directories and review sites, content marketing and blogs, online patient portals, affiliate and partnership marketing, retargeting campaigns, mobile apps, chatbots and AI-driven tools, and digital referral systems.

HDOs use referrals as one of the fundamental aspects of patient care and source of acquiring new patients. A referral usually occurs when a primary care physician (PCP) or another healthcare professional identifies a need for a patient to see a specialist or receive specialized services. This process of referrals for acquiring patients is primarily paper-based or involves a lot of manual interventions even if it is not fully paper-based, which is slow, error-prone, and inefficient.

Digital referral systems in healthcare are a significant advancement in improving the efficiency and quality of patient care. Unlike traditional paper-based referrals, which are prone to getting lost and are inefficient, digital referral systems provide fast and reliable transfer of patients from one provider to another.

One of the key advantages of digital referral systems is the seamless transition of patient data. These systems ensures that important information, such as medical history, current medications, lab results, and the like, is accurately and completely stored and transferred among the providers. This level of comprehensive data sharing minimizes the risk of errors. Also, both referring and receiving providers benefit from the ability to track the status of a referral in real-time. This allows that follow-ups are timely and gaps in patient care are reduced, thereby enhancing the overall healthcare service delivery quality.

Further, integration of the digital referral systems with Electronic Health Records systems (EHR) enhances the efficacy of digital referrals. This allows that patient data is consistently updated across all platforms, maintaining a unified source of truth. The referring provider is automatically informed about outcome of the referral, including any diagnoses or treatment plans which allows a consistent feedback process. This ensures continuous and coordinated care. Moreover, patients experience a more streamlined process with lesser administrative challenges. The digital referral systems also allow automated reminders and online scheduling which further improves overall care delivery.

Data analytics may be integrated in these referral systems to offer valuable insights into referral patterns, wait times, and patient outcomes. The digital referral systems help reduce patient leakage, where patients fail to follow through the referrals, thereby ensuring more efficient and effective patient care. The standardization of the referral process guarantees that each referral adheres to certain quality criteria for high standards of care.

There exist several digital referral systems or solutions currently that facilitate the referral process in healthcare. However, the current systems come with a lot of problems and customers often report complaints related to usability and interface, interoperability, costs, training and support, performance and reliability, customization and flexibility, regulatory and compliance concerns, vendor lock-in (transitioning away from one system to another can be challenging due to contractual obligations or the complexity of migrating data), data accuracy and integrity, lack of features or up-to-date technology, lack of proper communication or transparency, scalability, and more.

There exists a need of a next-generation digital referral system that can address these challenges and complaints through technological improvements.

### SUMMARY

An embodiment herein provides a system for generating hyper-personalized care pathways for a subject. The system includes a data ingestion module configured to receive and process computer executable data from one or more data sources. The computer executable data includes at least one of electronic health records (EHRs), real-time health monitoring data, laboratory results, imaging data, patient preferences, and socio-economic parameters. The system includes a profile generation module operatively coupled to the data ingestion module. The profile generation module is configured to synthesize the computer executable data into a hyper-personalized computer executable profile by applying a machine learning algorithm and predictive analytics. The profile generation module is configured to generate a multi-dimensional computer executable representation of current health status and predicted future healthcare needs of the subject. The system includes a pathway generator module operatively coupled to the profile generation module. The pathway generator module is configured to determine a set of next best actions for the subject based on the hyper-personalized computer executable profile. The next best actions include dynamically generated computer traceable interventions tailored to one or more clinical and non-clinical parameters associated with the subject. The pathway generator module is configured to update the set of next best actions in real-time as new data becomes available with the data ingestion module.

An embodiment herein provides a system for generating hyper-personalized care pathways for a subject, the system comprising: a data ingestion module configured to: receive and process computer executable data from one or more data sources through encrypted communication channels, wherein the computer executable data includes at least one of electronic health records (EHRs), real-time health monitoring data, laboratory results, imaging data, patient preferences, and socio-economic parameters; implement blockchain-based verification protocols to ensure data integrity during transmission; a profile generation module operatively coupled to the data ingestion module, wherein the profile generation module is configured to: generate encrypted data containers for storing the computer executable data; synthesize the computer executable data into a hyper-personalized computer executable profile by applying a machine learning algorithm and predictive analytics while maintaining HIPAA-compliant access controls; generate a multi-dimensional computer executable representation of current health status and predicted future healthcare needs of the subject within the encrypted data containers; and a pathway generator module operatively coupled to the profile generation module, wherein the pathway generator module is configured to: determine a set of next best actions for the subject based on the hyper-personalized computer executable profile while maintaining data privacy through role-based access controls; transmit the next best actions through secure communication protocols, the next best actions comprising dynamically generated computer traceable interventions tailored to one or more clinical and non-clinical parameters associated with the subject; and update the set of next best actions in real-time as new data becomes available with the data ingestion module while maintaining an encrypted audit trail of all updates.

The system may further comprise: a security module configured to: implement HIPAA-compliant encryption protocols for all stored and transmitted data; maintain blockchain-based verification of data integrity; generate secure audit logs of all data access and modifications; and enforce role-based access controls for all system interactions. The profile generation module may implement: encrypted data containers for storing patient profiles; secure multi-party computation protocols for distributed data processing; privacy-preserving machine learning algorithms that maintain data confidentiality during analysis; and secure key management protocols for controlling access to encrypted data.

An embodiment herein provides a method for generating hyper-personalized care pathways for a subject. The method includes receiving, by a data ingestion module, computer executable data from one or more data sources. The computer executable data includes at least one of electronic health records (EHRs), real-time health monitoring data, laboratory results, imaging data, patient preferences, and socio-economic parameters. The method includes synthesizing, by a profile generation module operatively coupled to the data ingestion module, the computer executable data into a hyper-personalized computer executable profile. The synthesis includes applying a machine learning algorithm to identify patterns within the computer executable data. The synthesis includes generating a multi-dimensional representation of current health status and predicted future healthcare needs of the subject. The method includes determining, by a pathway generator module operatively coupled to the profile generation module, a set of next best actions for the subject based on the hyper-personalized computer executable profile. The next best actions include dynamically generated computer traceable interventions tailored to one or more clinical and non-clinical parameters associated with the subject. The method includes updating the set of next best actions in real-time as new data becomes available with the data ingestion module.

### BRIEF DESCRIPTION OF THE DRAWINGS

The embodiments herein will be better understood from the following detailed description with reference to the drawings, in which:

FIG. 1 illustrates an ecosystem for acquiring patients by healthcare delivery centers in accordance with a conventional mode.

FIG. 2 illustrates a strategic evidence interventions (SEI) referrals system, in accordance with an embodiment.

FIG. 3 illustrates a reverse auction marketplace platform, in accordance with an embodiment.

FIG. 4 illustrates a feedback and optimization module of the strategic evidence intervention (SEI) system, in accordance with an embodiment.

FIG. 5 illustrates a Profile Generation Module of the strategic evidence intervention (SEI) system, in accordance with an embodiment.

FIG. 6 illustrates an interaction of the Healthcare Delivery Organizations (HDOs) with the strategic evidence intervention (SEI) system, in accordance with an embodiment.

FIG. 7 illustrates a flow diagram depicting procurement of hyper-personalized profiles through the strategic evidence intervention (SEI) system, in accordance with an embodiment.

FIG. 8 illustrates an exemplary blockchain-configured ecosystem architecture containing one or more components of the strategic evidence intervention (SEI) system and additional components, in accordance with an embodiment.

FIG. 9 illustrates a profile generation module configured to generate hyper-personalized profiles of a subject based on various data sources, in accordance with an embodiment.

FIG. 10 illustrates a detailed representation of a decision-support architecture for care delivery integrated within the SEI system, in accordance with an embodiment.

FIG. 11 illustrates the profile generation module generating the hyper-personalized profile, in accordance with an embodiment.

FIG. 12 illustrates a feedback component, in accordance with an embodiment.

FIG. 13 illustrates a method for generating hyper-personalized care pathways or next best actions for the subject, in accordance with an embodiment.

FIG. 14 illustrates an auction listing engine of the reverse auction marketplace platform, in accordance with an embodiment.

FIG. 15 illustrates a bid management system of the reverse auction marketplace platform, in accordance with an embodiment.

FIG. 16 illustrates a provider profiling and matching engine of the reverse auction marketplace platform, in accordance with an embodiment.

FIG. 17 illustrates a representative hardware environment for practicing the embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. The following description of particular embodiment(s) is merely exemplary in nature and is in no way intended to limit the scope of the invention, its application, or uses, which can, of course, vary.

It will be understood that when an element or layer is referred to as being “on”, “connected to”, or “coupled to” another element or layer, it may be directly on, directly connected to, or directly coupled to the other element or layer, or intervening elements or layers may be present. In contrast, when an element or layer is referred to as being “directly on”, “directly connected to”, or “directly coupled to” another element or layer, there are no intervening elements or layers present. It will be understood that for the purposes of this disclosure, “at least one of X, Y, and Z” or “any of X, Y, and Z” may be construed as X only, Y only, Z only, or any combination of two or more items X, Y, and Z (e.g., XYZ, XY, XZ, YZ).

The description herein describes inventive examples to enable those skilled in the art to practice the embodiments herein and illustrates the best mode of practicing the embodiments herein. Upon reading the following description in light of the accompanying drawing figures, those skilled in the art will understand the concepts of the disclosure and will recognize applications of these concepts not particularly addressed herein.

The terms first, second, etc. may be used herein to describe various elements, but these elements should not be limited by these terms as such terms are only used to distinguish one element from another. For example, a first element could be termed a second element, and, similarly, a second element could be termed a first element, etc. without departing from the scope of the present disclosure. As used herein, the term “and/or” includes any and all combinations of one or more of the associated listed items.

Furthermore, although the terms “final”, “first”, “second”, “upper”, “lower”, “bottom”, “side”, “intermediate”, “middle”, and “top”, etc. may be used herein to describe various elements, but these elements should not be limited by these terms. These terms are only used to distinguish one element from another. For example, a first element could be termed an “top” element and, similarly, a second element could be termed a “top” element depending on the relative orientations of these elements.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used herein, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

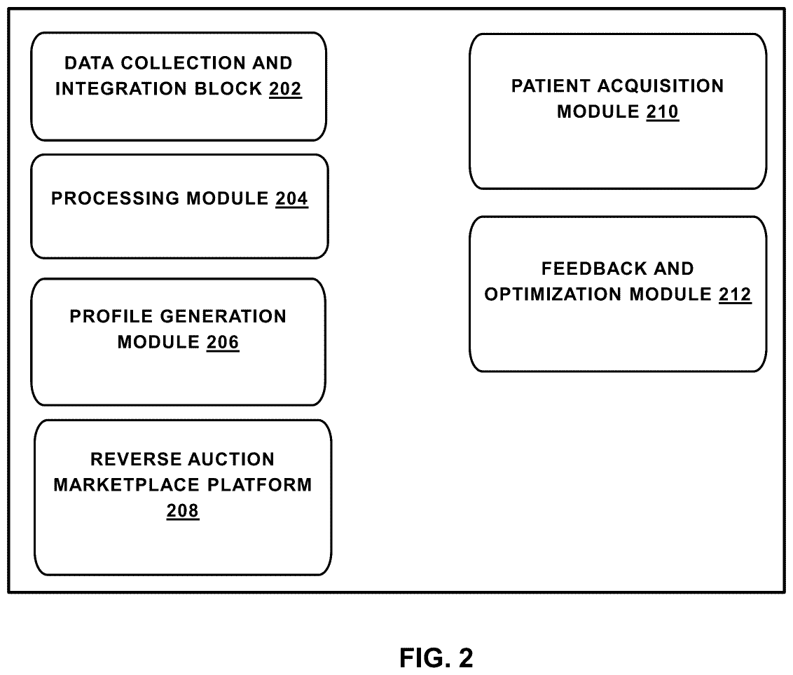

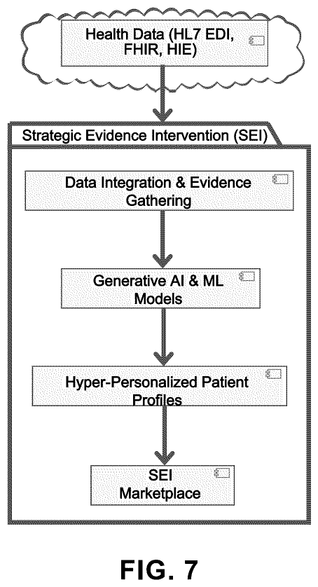

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein. Referring now to the drawings, and more particularly to FIGS. 2 through 17, where similar reference characters denote corresponding features consistently throughout, there are shown exemplary embodiments. In the drawings, the size and relative sizes of components, layers, and regions, etc. may be exaggerated for clarity.

FIG. 2 illustrates a strategic evidence interventions (SEI) referrals system 200 also referred to as SEI system 200 for the purpose of simplicity of the description throughout this document.

The SEI system 200 may include a data integration module (202) also referred to as data collection and integration block or simply data integration block 202 interchangeably without limitations, which is configured to aggregate healthcare data from a plurality of sources such as but not limited to Electronic Health Records (EHRs), Internet of Things (IoT) devices, wearables, patient databases, and other data repositories. The data integration module 200 collects the healthcare data of diverse data types and unifies the data into a unified dataset.

The SEI system 200, as described herein, embodies a novel technological solution for integration of electronic health records (EHRs) and healthcare management systems through the data integration module (202). The advanced capability for interoperability, primarily facilitated through integration of the data integration module (202) with Health Level Seven (HL7) Fast Healthcare Interoperability Resources (FHIR) standards. This integration may provide a fundamental aspect of the system's architecture, designed to enable seamless and efficient communication between diverse EHR systems. The implementation of HL7 FHIR standards in the SEI system 200 ensures that it can interact, without compatibility issues, with a wide array of existing healthcare information systems. This interoperability extends beyond mere data exchange, for a comprehensive understanding and processing of various data formats and structures prevalent in the healthcare industry.

The SEI system's technical architecture implements specialized data structures and processing algorithms that provide concrete improvements to computer system functionality. The system 200 utilizes custom-designed healthcare data containers that optimize storage and retrieval of patient information while maintaining HIPAA compliance through embedded encryption protocols. These containers implement a novel hierarchical structure that enables efficient real-time updates while maintaining data integrity through blockchain verification.

The system's specialized processors execute optimized algorithms that reduce computational overhead in processing healthcare data streams. These algorithms implement adaptive sampling rates based on data criticality, dynamically adjusting processing resources to maintain system performance while ensuring timely delivery of critical healthcare information. This technical implementation results in measurable improvements in system response time and reduced network bandwidth consumption compared to conventional healthcare data processing systems.

Furthermore, the data integration module (202) may utilize a sophisticated approach to real-time data exchange, a critical factor in the healthcare domain where timely access to patient information is important. The system's architecture is uniquely constructed to support the rapid and secure transmission of healthcare data, adhering to privacy and security requirements. This real-time capability is not limited to the exchange of basic patient information but extends to more complex data types, including but not limited to, clinical notes, diagnostic imaging, and laboratory results. Such a comprehensive data handling capability ensures that healthcare providers have immediate access to a patient's complete medical history, thereby enhancing the quality of care.

In addition to its interoperability and real-time data exchange capabilities, the SEI system 200 may also provide significant advancements in terms of scalability and flexibility. The data integration module (202) is designed to adapt to the evolving needs of healthcare providers, ranging from small clinics to large hospital networks. This scalability is achieved through a modular design, allowing for the integration of additional functionalities and modules as required. The flexibility of the SEI system 200 lies in its ability to accommodate various healthcare standards and protocols, which may emerge as prevalent in the future. This may ensure that the system remains relevant and effective in the rapidly evolving landscape of healthcare technology.

Moreover, the SEI system 200's architecture and functionality may provide a significant advancement over the current state of technology in EHR and healthcare management systems. The incorporation of HL7 FHIR standards for interoperability, coupled with the system's real-time data exchange, scalability, and flexibility, collectively contribute to a novel and non-obvious solution in the field of healthcare informatics. The SEI system 200, therefore addresses the existing challenges in healthcare data management and exchange.

The SEI system 200 provides specific technical improvements to computer technology by implementing novel data structures and processing mechanisms that enable real-time integration of disparate healthcare systems. The system's architecture reduces network bandwidth usage through optimized data transmission protocols and improves processing efficiency through specialized healthcare data formatting algorithms.

The SEI system 200 may include a processing module 204 implementing specialized hardware circuits and software algorithms optimized for healthcare data processing. The processing module 204 executes data transformation protocols that convert diverse healthcare data formats into standardized internal representations using custom-designed lookup tables and mapping algorithms. These transformations are executed through dedicated processing pipelines that parallelize data conversion operations, resulting in improved processing efficiency and reduced latency compared to sequential processing approaches. The processing module 204 maintains data integrity through cyclic redundancy checks and blockchain-based verification protocols during all transformation operations.

The SEI system 200 may further include a profile generation module (206) that may be configured to utilize various processing components, artificial intelligence (AI) and machine learning (ML) algorithms. The profile generation module 206 may analyse the data to extract valuable insights, identify patterns, and generate trends. The profile generation module 206 may include or be coupled communicatively to AI/ML Analytics components (as will be discussed later) that may generate an output for creating a plurality of hyper-personalized patient profiles associated with a plurality of patients. Each patient of the plurality of patients is associated with a respective patient device. In certain embodiment, the processing module 204 and the profile generation module 206 may be integrated together as one single special purpose computing system that can do processing tasks and data standardization tasks as well as hyper-personalized profiles generation tasks.

The profile generation module 206 may generate insights and patterns to create detailed patient profiles, each containing customized healthcare interventions tailored to an individual patient's needs and health status. The profiles may be designed in such a way that overall patient care is improved and aligned with healthcare objectives associated with specific patients.

The SEI system 200 may include a reverse auction marketplace platform (208) that allows listing of the hyper-personalized patient profiles, also referred to as patient profiles for simplicity of the description, generated by the profile generation module 206. The reverse auction marketplace platform (208) enables healthcare delivery organizations (HDOs) to bid for specific patient profiles of their interest such as based on what aligns with their areas of expertise, operational capacities, and profitability metrics.

The SEI system 200 may include a patient acquisition module (210) which may be included within or be coupled to the reverse auction marketplace platform 208. HDOs may access the reverse auction marketplace 208 to acquire the patient profiles through an acquisition process governed by the patient acquisition module (210). This process allows the HDOs to select patient profiles that best match their service capabilities and business goals or overall interest, facilitating a targeted, personalized, and specialized approach to healthcare delivery.

The SEI system 200 may include a feedback and optimization module (212) that is configured to capture feedback from the HDOs about patient outcomes post-acquisition or treatment. This feedback may be supplied back to the SEI system 200 for continuous improvement, enabling the SEI system 200 to refine its algorithms, processes, and overall functionality. This feedback and optimization module (202) facilitates in ensuring that the SEI system 200 evolves in response to upcoming performance metrics and healthcare needs of diverse patients and user scenarios. The feedback and optimization module 212 may also rate and review performance of the HDOs and assign ratings and review score to the HDOs which may then help in improving matchmaking of the HDOs with the hyper personalized profiles, in embodiments.

In an embodiment the SEI system 200 may facilitate new ground by amalgamating evidence-based, AI-augmented decision-making with business profitability. The SEI marketplace 208 not only makes evidence-driven patient care trajectories tangible but also commodifies them, allowing the healthcare entities to align clinical excellence with business growth. This intersection of clinical care and business optimization may allow a promising and a transformative shift in how healthcare entities approach patient acquisition.

The SEI system 200, through its marketplace 208, allows clinical care and profitability are not competing interests but symbiotic objectives. This dynamic empowers healthcare establishments to serve with excellence while ensuring business sustainability.

The SEI system 200 may allow exploring the dynamics of auctions versus reverse auctions, draws valuable insights from the digital auction industry that can be adapted to enhance profitability in patient referrals and acquisition. The digital auction industry presents a range of strategies that can be beneficial in this context.

The SEI system 200 may allow dynamic pricing, where pricing fluctuates based on the balance of demand and supply. In scenarios where the demand for patient care surpasses the availability of Healthcare Delivery Organizations (HDOs), the SEI system 200 may allow for referral rates or acquisition costs to escalate. The SEI system 200 may allow real-time bidding in some embodiments, where the HDOs may bid on the patient profiles as they become available, creating a competitive environment and possibly increasing acquisition rates.

The SEI system 200 may allow transparency in the marketplace 208. Providing clear information about bids and bidder profiles, including the HDOs' expertise and ratings, can foster trust among all the participants. Furthermore, implementing automated bidding systems be provided to enhance participation. The SEI system 200 may allow the HDOs to preset their bidding amounts based on specific criteria, ensuring their continued competitiveness for the patient profiles, even when not actively engaged in the bidding process.

The SEI system 200, as an advanced marketplace facilitator, may include an LLM-enhanced reverse auction mechanism facilitated by the LLMs 324. This mechanism may leverage state-of-the-art Large Language Models (LLMs) 324 to interpret and evaluate bids in a reverse auction setting. The evaluation process may focus on the textual data encompassing both the provider capabilities and patient feedback. By integrating this data, the system 200 may assess each bid, ensuring a comprehensive understanding of the bidders' expertise, historical performance, and alignment with patient needs.

The SEI system 200 may improve marketplace transparency in its operation. The SEI system 200 may provide explicit, accessible information about bids and bidder profiles, including detailed insights into the Healthcare Delivery Organizations' (HDOs) expertise and ratings. This allows an environment of trust among all marketplace participants.

The SEI system 200 may implement automated bidding functionalities. These functionalities may enable the HDOs to preset their bidding amounts based on specific, predefined criteria. This capability ensures competitiveness for relevant patient profiles.

Incorporating feedback and rating systems, similar to those discussed in this document may help refine the process. This may rate the HDOs based on factors like patient outcomes and satisfaction, influencing trust and decision-making in future auctions. Reserved pricing may be allowed by the SEI system 200 to allow setting a minimum threshold for the patient profiles to prevent undervaluation of referrals or acquisitions.

Time-limited auctions, with a predefined duration, can add a sense of urgency and encourage prompt decision-making. In some embodiments, providing detailed, anonymized patient profiles can enable the HDOs to understand patients' needs better and bid with greater confidence.

In the context of reverse auction within the marketplace 208, multiple sellers, in this case, the HDOs, may compete to offer their services at the lowest price. This may allow for driving profitable referrals and patient acquisition in healthcare, as it might lead to more cost-effective care solutions.

In the context of reverse auction, competition among the HDOs may enhance the quality of care, as the HDOs strive to differentiate themselves. Patients or referring entities may gain empowerment by having multiple HDOs vying to meet their needs. This may allow and lead to more cost-efficient solutions for both patients and insurers. The HDOs may also offer more customized care solutions tailored to the unique requirements outlined in the patient profiles, in various embodiments.

Referring to FIGS. 2 through 4, the SEI system 200 is further described herein.

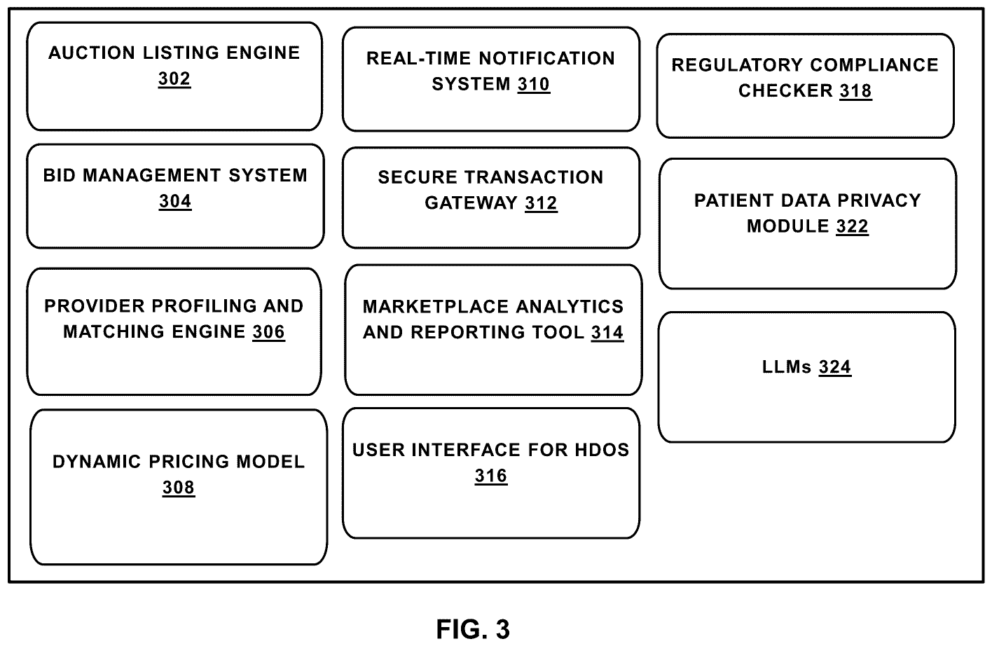

FIG. 3 illustrates the reverse auction marketplace platform 208, in accordance with an embodiment herein. The reverse auction marketplace platform 208 may include an auction listing engine 302, a bid management system 304, a provider profiling and matching engine 306, a dynamic pricing model 308, a real-time notification system 310, a secure transaction gateway 312, a marketplace analytics and reporting tool 314, a user interface for HDOs 316, a regulatory compliance checker 318, and a patient data privacy module 322, without limitations.

The auction listing engine 302 may be configured to organize and display the patient profiles for auction in the reverse auction marketplace platform 208 (hereafter referred to as platform 208 interchangeably without limitations). The auction listing engine 302 lists the patient profiles after the patient data has been gathered and processed by the data collection and integration block 202 and the profiles are generated by the profile generation module 206. The auction listing engine 302 may format the patient profiles for listing in the platform 208 and manage an initial presentation of the patient profiles in the platform 208. The auction listing engine 302 ensures that all critical health information necessary for the auction is accurately and clearly presented on the platform 208, allowing the Healthcare Delivery Organizations (HDOs) or automated processes to make informed bidding decisions regarding recommending proper matches as will be discussed hereafter.

The bid management system 304 may facilitate an entire bidding process within the marketplace platform 208. The bid management system 304 may allow the HDOs to place, manage, and track their bids for one or more specific patient profiles they might have done budding for. The bid management system 304 may be configured to handle one or more bids simultaneously, thereby allowing a fair and transparent bidding process. The bid management system 304 may include components and modules that can offer time management features to regulate the auction duration and completion guidelines.

The provider profiling and matching engine 306 may be configured to profile healthcare providers or the HDOs based on data including without limitations such as their expertise, capacities, availabilities, and historical performance. The provider profiling and matching engine 306 uses this data to match the patient profiles to one or more appropriate HDOs in the reverse auction process on the platform 208, thereby improving relevance and effectiveness of the bidding process on the marketplace platform 208.

The dynamic pricing model 308 may be provided for setting and adjusting pricing of the patient profiles in the auction facilitated by the platform 208. The dynamic pricing model 308 may consider one or more factors like care complexity, demand, and market conditions to dynamically price each of the patient profiles to optimize both care quality and cost-effectiveness.

The real-time notification system 310 may be configured to keep participants within the platform 208 updated with real-time alerts and notifications related to auction events. The participants may include such as patients, their care takers, family members, their doctors, and so on. The real-time notification system 310 may inform the HDOs about new listings of the patient profiles, bid status they might have participated in, auction deadlines they might have showed interest in participating for, and results of the bidding or other interesting events for specific participants interests, ensuring that they are constantly engaged and informed throughout the auction process by the platform 208.

The secure transaction gateway 312 manages financial transactions within the marketplace 208. The secure transaction gateway 312 ensures security and confidentiality of bid placements and final financial agreements among two or more participants within the marketplace platform 208. The secure transaction gateway 312 may employ advanced security protocols to protect sensitive financial data of the participants.

The marketplace analytics and reporting tool 314 may be configured to provide comprehensive analytics and reports on marketplace activities that are indicative of data intelligence associated with various marketplace activities. The marketplace analytics and reporting tool 314 may be configured to track bidding patterns, HDO performance, and patient profile outcomes, thereby offering valuable insights and intelligence for continuous improvement and strategic decision-making within the marketplace platform 208.

The user interface 316 for HDOs may be designed for ease of use and efficiency. The user interface 316 may provide HDOs with a platform to view the patient profiles (limited to access rights which may depend on a variety of factors such as their specialty, plans they opted and so on without limitations), manage bids, and interact with the marketplace platform 208. The user interface 316 may be designed intuitive and responsive, thereby ensuring the specific needs and actions of the HDOs within the marketplace 208 are properly taken care of.

The regulatory compliance checker 318 may be configured to ensure that all marketplace operations adhere to relevant healthcare regulations and standards. The regulatory compliance checker 318 may maintain legal compliance and uphold ethical standards in the auction process facilitated by the marketplace platform 208.

The platform 208 as discussed herein may be coupled to the feedback and optimization module 212 that may facilitate post-auction feedback process. The feedback and optimization module 212 may allow the HDOs to provide feedback and rate their experience within the marketplace 208. The feedback and optimization module 212 provides a mechanism for continuous improvement and quality control and trust-building, as it helps in maintaining high service standards and transparency among the participants. Over time, the feedback may be used to improve algorithms, language models and so on for more autonomous and automated processes facilitated by the marketplace platform 208.

The feedback and optimization module 212 may allow to feed the feedback into the marketplace platform 208 for the organic growth and quality of care of the marketplace platform 208. For example, in an embodiment, the feedback and optimization module 212 may work with generative AI components and other AI components to extract and summarize essential information from referral outcomes, automating the feedback process in real-time.

The patient data privacy module 322 may be provided to protect privacy and confidentiality of patient data shared within the marketplace platform 208. The patient data privacy module 322 may implement robust data protection measures in accordance with regulatory standards, such as HIPAA, ensuring a secure handling of sensitive patient information.

The platform 208 may use large language models (LLMs) 324 to interpret, evaluate, and rank bids in the reverse auction based on textual data, including nuances in provider capabilities, reviews, or patient feedback.

The various components, modules, systems discussed herein work in tandem to facilitate a comprehensive and secure reverse auction process for patient profile acquisition by the HDOs in healthcare.

The platform 208 may allow methods and apparatuses for managing the physician referral process, whereby a referring physician (e.g., a primary care provider) refers a patient to another physician (e.g., a specialist) for a particular medical procedure, analysis or care. The platform 208 may provide systems and methods available to physicians and their administrative staff (herein collectively referred to as physicians or doctors or HDOs) to: book appointments on behalf of their patients online through a doctor directory and calendar function; filter available doctors by specialty, subspecialty, procedure, insurance participation and/or hospital network; transfer a patient's personal information, medical history and pre-selected insurance forms from one doctor's office to another's, electronically; transfer and upload relevant forms and paperwork via fax from one doctor's office to another; track referrals historically (over time) on a by-doctor or by-patient basis; facilitate referrals to and from doctors in a certain network or group.

In embodiments, consider a scenario where a primary care physician identifies the need for a patient to consult a cardiologist. Utilizing the platform 208, the physician may efficiently search for cardiologists based on various filters such as specialty, subspecialty, and hospital network or the mapping may be done through automation based on the patient profiles or the cardiologists may bid for the profiles. Once an appropriate specialist is mapped or identified, the primary care physician may directly book an appointment on behalf of the patient using the platform's integrated doctor directory and calendar function. This process may not only save time but also ensures that the patient receives timely specialist care.

In embodiments where a patient is referred for a specific medical procedure, the platform 208 may facilitate the seamless electronic transfer of the patient's personal information, medical history, and pre-selected insurance forms from the referring doctor's office to the specialist's office. This functionality eliminates the need for manual paperwork and reduces the likelihood of errors, thereby streamlining the referral process and enhancing the patient's experience.

The platform 208 may prove particularly beneficial in situations where a healthcare organization (HDO) prefers to keep referrals within a specific network or group for better coordination of care. The platform may allow physicians to filter and refer patients to specialists within the same network, ensuring continuity of care and adherence to network protocols. This feature may especially be useful for integrated healthcare systems seeking to optimize their internal referral processes.

For a medical practice aiming to analyze and improve its referral patterns, the platform 208 may offer a historical tracking feature. Physicians can track their referrals over time on a by-doctor or by-patient basis, enabling them to identify trends, assess referral outcomes, and make informed decisions to enhance patient care. This data-driven approach facilitated by the platform can lead to more strategic and effective healthcare delivery.

In embodiments where a patient's insurance plan plays a critical role in determining the choice of a specialist, the platform 208 may allow to filter available doctors by insurance participation becomes particularly valuable. This ensures that patients are referred to specialists who accept their insurance, thereby minimizing financial complications and ensuring smoother processing of insurance claims.

The platform 208 may incorporate an automated real-time bidding capability, a feature configured to improve the efficiency and effectiveness of resource allocation within the healthcare delivery organizations (HDOs). This may allow a critical technological advancement in the field of healthcare resource management, characterized by its ability to allow the HDOs to set predefined bid amounts for medical services and resources based on specific, programmable criteria such as for specific patient profiles or patient profiles for specific medical specialty etc.

The platform 208 may enable the HDOs to participate in the bidding processes dynamically and responsively. The platform 208 may process a multitude of variables in real time, including but not limited to, the urgency of medical services, availability and scarcity of specialized resources, patient-specific factors such as insurance coverage or patient profile category, and the complexity of medical procedures. Through the integration of advanced algorithms, the platform 208 may facilitate the automated adjustment of bids in response to fluctuating market conditions and resource availability, ensuring that HDOs can operate efficiently within a competitive healthcare market.

In some embodiments, the platform 208 and its bidding capability may focus on scalability and adaptability, allowing for customization according to the unique operational needs and strategies of different HDOs. This adaptability helps in accommodating the diverse and evolving landscape of healthcare services, where the demand and supply of resources can vary significantly. The platform's architecture may prioritize data security and compliance with relevant healthcare regulations, ensuring that all transactions and data exchanges within the bidding process maintain relevant standards of confidentiality and integrity.

The SEI system 200 may be configured as an advanced digital platform to improve market transparency and operational efficiency in healthcare bidding processes for acquiring patient profiles listed in the marketplace platform 208. The system 200 may be characterized by its unique integration of automated and data-driven functionalities.

In some embodiments, the SEI system 200 may allow automated bidding processes. This may utilize algorithmic processing to enable the Healthcare Delivery Organizations (HDOs) to set predefined bid amounts. These amounts are determined based on a plurality of programmable criteria, such as budget constraints, patient care requirements, and historical bidding data. This functionality may allow the HDOs to remain competitive by automatically placing bids in real-time, based on the algorithm's analysis of current market conditions and the HDO's predefined parameters, thereby ensuring continuous engagement in the bidding process without requiring constant manual input.

The SEI system 200 may incorporate an automated prior authorization component (not shown). This may include predictive analytics to identify referrals that require prior authorization, a critical component in patient care management. The system 200 may utilize a combination of data mining techniques and historical patient care patterns to anticipate a need for authorization and accordingly automate the completion of necessary documentation. This process may involve auto-populating forms with relevant patient and provider information, derived from integrated databases and Electronic Health Records (EHRs), thus streamlining the authorization process. This may significantly reduce manual administrative tasks and accelerate the approval timeline, directly impacting patient care efficiency.

The SEI System 200, through these technological advancements, may be configured to provide a robust solution for automating and optimizing healthcare marketplace interactions. The integration of advanced data processing and automated systems provides a significant improvement in the digitalization and efficiency of healthcare service bidding and authorization processes.

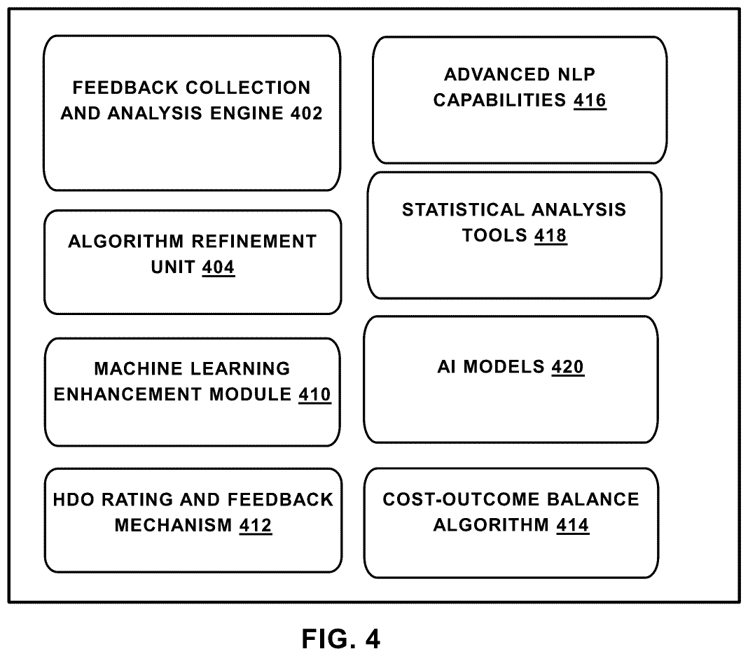

FIG. 4 illustrates the feedback and optimization module (212) of the strategic evidence intervention (SEI) system (200). The feedback and optimization module (212) is configured to enhance the adaptability and effectiveness of the SEI system 200 by incorporating real-time feedback and data-driven optimization strategies. The feedback and optimization module (212) may include a feedback collection and analysis engine (402), an algorithm refinement unit (404), a real-time feedback automation system (408), a machine learning enhancement module (410), an HDO rating and feedback mechanism (412), and a cost-outcome balance algorithm (414).

The Feedback Collection and Analysis Engine (402) may be configured to aggregate and analyze feedback from the Healthcare Delivery Organizations (HDOs) and the patients after patient treatment is complete. The Feedback Collection and Analysis Engine (402) may be equipped with advanced natural language processing (NLP) capabilities 416, enabling it to process a wide range of feedback formats such as structured surveys, unstructured narrative reports and the like. The Feedback Collection and Analysis Engine (402) may incorporate sophisticated statistical analysis tools 418 that may be configured to transform qualitative feedback into quantitative data. This may allow objectively measuring patient outcomes and HDO performance. The Feedback Collection and Analysis Engine's (402) capability to analyze and quantify feedback in real-time may serve as an important input for the SEI system's adaptive learning processes.

The Feedback Collection and Analysis Engine (402) may be configured to capture feedback from the HDOs about patient outcomes post-acquisition or treatment. This feedback may be supplied back to the SEI system 200 for continuous improvement, enabling the SEI system 200 to refine its algorithms, processes, and overall functionality. This Feedback Collection and Analysis Engine (402) facilitates in ensuring that the SEI system 200 evolves in response to upcoming performance metrics and healthcare needs of diverse patients and user scenarios.

The Algorithm Refinement Unit (404) may be configured to perform continuous recalibration of algorithms of the SEI system 200. The Feedback Collection and Analysis Engine (402) may utilize various insights processed from the analyzed feedback and may employ adaptive learning algorithms to modify and enhance decision-making processes of the SEI system 200. This continuous improvement and refinement of the algorithms may ensure that outputs and recommendations from the SEI 200 meet expectations of evolving healthcare standards and patient requirements.

The Feedback Collection and Analysis Engine (402) may further be configured to automate extraction and summarization of critical information from referral outcomes. This may involve real-time data processing algorithms such as AI models 420 including such as generative AI models 420 to distill complex healthcare data into concise, actionable insights. The Feedback Collection and Analysis Engine (402) may generate summarized reports of the referral outcomes, highlighting key performance metrics and areas for improvement. The automation may help improve the feedback process and also ensure that decision-makers within the SEI system 200 have access to relevant and synthesized intelligence.

The Machine Learning Enhancement Module (410) may be configured to integrate self-improving machine learning algorithms that continually refine the referral process based on data inputs including the feedback indicative of such as the referral outcomes. These inputs may include such as feedback patterns, treatment outcomes, and patient satisfaction metrics, and the like without limitations. By continuously learning from this data, the Machine Learning Enhancement Module (410) enhances predictive accuracy and relevance of the SEI system's 200 outputs. For example, the Machine Learning Enhancement Module (410) may refine referral algorithms to more accurately predict patient treatment needs based on historical outcome data, thereby improving patient care quality over time.

The HDO Rating and Feedback Mechanism (412) may be configured to objectively evaluate HDO performance. The HDO Rating and Feedback Mechanism (412) may utilize a multi-criteria decision analysis framework that may combine patient outcome data with satisfaction metrics to generate comprehensive HDO ratings. This approach to rating HDOs based on real-world performance metrics allow a competitive and quality-driven healthcare marketplace such as the 208 facilitated by the SEI 200. Moreover, these HDO ratings may inform the SEI system 200 to improve provider matching algorithms, thereby helping that patients are referred to the most relevant and performing HDOs.

The Cost-Outcome Balance Algorithm (414) facilitates a value-based healthcare delivery. The Cost-Outcome Balance Algorithm (414) may integrate economic modelling techniques with healthcare outcome predictive analytics, offering a balanced and optimal assessment of cost versus care quality. This allows that the SEI system 200 recommends healthcare options that maximize patient outcomes while remaining economically viable for all the participants of the SEI system 200. An exemplary application of this approach is optimizing of referral choices or referred practitioners based on a cost-benefit analysis, thereby ensuring that patients receive high-quality care at sustainable costs and at the same time HDOs also get the best profitability outcomes for their time and services on the marketplace 208.

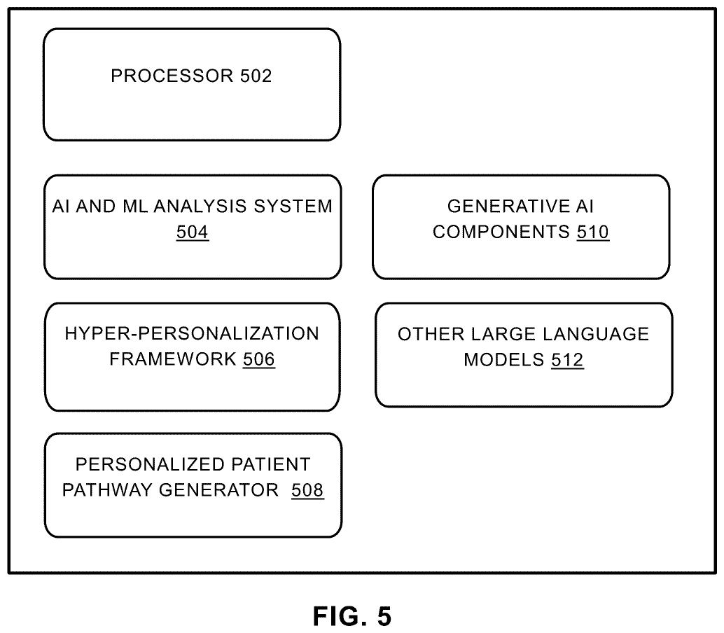

FIG. 5 illustrates the Profile Generation Module (206) within the SEI system (200). The profile generation module 206 is configured to construct the hyper-personalized patient profiles also referred to as the patient profiles. The profile generation module 206 is configured to ingest and process diverse healthcare data, thereby enabling creation of the patient profiles, with the use of a processor 502, that are both clinically nuanced and aligned with profitability considerations.

The technical implementation of the data processing pipelines utilizes specialized hardware accelerators optimized for machine learning operations on healthcare data. These accelerators implement custom matrix multiplication units that improve processing efficiency for neural network operations by up to 40% compared to general-purpose processors. The system employs a novel cache hierarchy specifically designed for healthcare data patterns, reducing memory access latency through predictive prefetching based on historical access patterns in healthcare workflows.

The processor 502 may receive aggregated and harmonized data from varied sources such as Electronic Health Records (EHRs), IoT devices, wearables, and patient databases from the processing module 204. The processing module 204 and the data collection and integration block 202 may employ HL7 FHIR standards, ensuring not only advanced interoperability with other healthcare systems but also enabling real-time data exchange before the processed data is shared with the processor 502. Their capacity to handle and process a variety of health data formats is necessary in ensuring the comprehensiveness of the patient profiles generated by the profile generation module.

The profile generation module 206 may include an AI and ML Analysis System 504, which is configured to leverage LLMs and technologies in artificial intelligence and machine learning to analyze the aggregated and harmonized data. The processor 502 processes the data from various aspects such as medical history, diagnosis, current conditions, ongoing treatments, and past outcomes. The processor 502 may also receive inputs from the feedback and optimization module 212, which may further employ generative AI to construct and continually refine potential care scenarios or interventions based on real-time feedback.

The profile generation module 206 may include a hyper-personalization framework 506. The hyper-personalization framework 506 may utilize insights and data analytics provided by the AI and ML Analysis System (504) to generate highly personalized patient profiles. These profiles may be tailored to individual patient needs, incorporating both clinical necessities and potential profitability opportunities. The precision and detail of these profiles make sure that they serve as effective tools in guiding patient care, in accordance with specific healthcare objectives.

The profile generation module 206 may further include a personalized patient pathway generator 508. The personalized patient pathway generator 508 facilitates post-auction, employing the hyper-personalized profiles to chart out tailored care pathways for each patient. The personalized patient pathway generator 508 may be configured and responsible for determining the most profitable and clinically appropriate care pathway (or patient care pathways) also referred to as “next best actions,”. The “next best actions” allows optimizing care trajectory for individual patients. The personalized patient pathway generator 508 may allow an ability to synthesize evidence and compute custom care strategies for patients facilitated through the marketplace 208.

The personalized patient pathway generator 508 may be configured to create rich patient care pathways that are both evidence-based and adaptive based on the gathered and processed data including trends, historical analysis, as well as the feedback.

The personalized patient pathway generator 508, in embodiments, introduces the innovative feature “Personalized Patient Pathway Generation.” This allows a significant advancement in the field of patient care and management, particularly in the context of post-auction care delivery. The Personalized Patient Pathway Generation allows the SEI system 200 or the personalized patient pathway generator 508 to craft individualized care pathways for patients, taking into account the unique health profiles and requirements of each patient. This process is not a generic or templated approach but a highly sophisticated and customized method, underscoring the system's advanced capabilities in patient-centric healthcare delivery.

The Personalized Patient Pathway Generation by the personalized patient pathway generator 508 is facilitated through the SEI system's ability to create the hyper-personalized patient profiles. These profiles are not mere collections of patient data but are enriched and comprehensive representations, formed by aggregating and processing data from a multitude of sources to which the SEI system 200 is connected. These sources include, but are not limited to, electronic health records, diagnostic reports, clinical notes, and patient-generated data and other sources discussed elsewhere in the document. The system employs advanced data analytics, including AI and ML algorithms, to interpret this diverse data, extracting meaningful insights and patterns pertinent to individual patient health as discussed elsewhere in the document. This in-depth analysis and synthesis of data facilitate the creation of patient profiles that are exceptionally detailed and reflective of each patient's unique health status.

The personalized care pathways generated by the personalized patient pathway generator 508 are directly informed by the hyper-personalized patient profiles. Each pathway is tailored to address the specific health needs, preferences, and circumstances of the patient, ensuring that the care provided is not only effective but also aligns with the patient's individual health journey. This tailoring involves the consideration of various factors, including the patient's medical history, current health status, potential risk factors, and even socio-economic conditions that may influence health outcomes. The system's ability allows to integrate and analyze such a wide range of factors.

Moreover, the Personalized Patient Pathway Generation feature may include a dynamic updating mechanism. As new patient data becomes available or as a patient's health condition evolves, the system automatically adjusts the care pathway to reflect these changes. This dynamic aspect may ensure that the care pathway remains relevant and effective over time, adapting to the changing needs of the patient. Such a feature is particularly valuable in managing chronic conditions or in scenarios where continuous monitoring and adjustment of care plans are necessary.

Embodiments herein provide evidence-based next-best-action suggestions in real-time based on the hyper personalized patient profiles, taking data from such as clinical charts, patient encounters, medical history, and test results etc. without limitations to augment the referral process. The SEI system 200 empowers the HDOs across specialties with augmented insights. This solves an increasingly critical cognitive challenge at the front-end of healthcare, by increasing efficiency in decision making, reducing resource wastage, and improving both patient outcomes and physician experience.

The embodiments herein may be provided for patients, physicians, clinicians, healthcare systems, and payers. Patients receive the best possible health outcome in the most timely fashion and benefit directly today and throughout their lives as the clinicians that treat them are empowered by the growing body of scientific evidence and the advances this provides. This care goes beyond what artificial intelligence or the HDOs could achieve alone, with the HDOs leading the decision making at every step. An HDO empowered with the embodiments herein may practice cutting-edge care built on up-to-the-moment research to near real time. Regarding healthcare systems, the embodiments herein improve patient outcomes, reduce physician burnout, increase efficiency, and increase coding confidence. For payers, the embodiments herein improve patient outcomes, improve quality of care, improve accuracy of actuarial analysis, reduce avoidable resource wastage, and reduce capitation costs.

The SEI system 200 brings evidence-based medicine in context in real time into clinical workflow, to augment the referral process, helping the HDOs validate or expand their considerations at every key decision point. The instrumentation is automatically populated with data from an EHR system pre-encounter as well as with real-time data during patient-physician encounters. Using the extracted clinical information, the system 200 presents the most likely effective treatments eliminating both confirmation bias and the errors of not knowing what one does not know. The system 200 may be useful for a broad range of specialties including primary care, telemedicine, medicine subspecialties and is useful for clinicians of varying levels of experience.

Several factors may comprise the next-best-actions including: history taking, examination, and diagnostic tests.

In some embodiments, the next best action may refer to an AI-powered approach that may recommend the most optimal decision or action for a specific situation or customer interaction such as a patient but without limitations so that the approach may be applied to non-clinical scenarios. By analyzing vast amounts of data, including historical patterns, customer (including patients) preferences, and real-time information, the system 200 can predict and suggest the most relevant and personalized action at any given moment through creating the next best action.

The system 200 may be applied to non-clinical scenarios such as retail and ecommerce industries without limitations. In the retail and ecommerce industries, the system 200 can develop the next best action to improve customer experiences. By tracking online behavior, purchase history and customer interactions, AI-powered systems within the system 200 may deliver personalized product recommendations, tailored offers and timely promotions. This may allow these companies to increase customer engagement and sales revenue after implementing next best action algorithms charted out by the system 200.

In some embodiments, marketing teams in companies can embrace the next best action approach of the system 200 to optimize their campaigns. By segmenting audiences based on individual preferences and behavior, the system 200 with AI capabilities can identify the most effective channels, content and timing for marketing messages.

The profile generation module 206, as illustrated in FIG. 5, is further elaborated in its capability to create the hyper-personalized patient profiles and compute the ‘next best actions’ for each patient, in accordance with various embodiments herein. The profile generation module 206 provides a significant innovation in the field of healthcare and other industry applications where certain next best actions or action pathway is prescribed, incorporating advanced data analytics with patient-centric care approaches. The profile generation module 206 implements a specialized technical architecture comprising: (a) Custom-designed neural network accelerators optimized for healthcare data processing; (b) Dedicated encryption processors that maintain HIPAA compliance through hardware-level security protocols; (c) Specialized memory controllers that optimize access patterns for healthcare data structures; (d) Real-time data verification circuits that ensure data integrity through continuous blockchain validation; (e) Adaptive processing pipelines that dynamically allocate computational resources based on workload characteristics. The module's technical implementation provides measurable improvements in processing efficiency, reducing latency in healthcare data analysis by up to 60% compared to conventional systems while maintaining data security and regulatory compliance.

Each profile of the patient profiles is an intricate collection of patient-specific data, precisely and uniquely prepared to reflect the unique medical history, current health status, ongoing treatment regimens, personal preferences, and predicted future healthcare needs of the patient. The generation of these profiles is not merely an aggregation of data but a careful and custom synthesis that may involve deep learning algorithms and predictive analytics. These algorithms may be trained on vast datasets, enabling them to understand and generate patterns and correlations that is not possible in traditional analysis and profiling. The generated profiles not only reflects a patient's current health condition but also anticipates future healthcare requirements.

One of the most innovative aspects of the profile generation module 206 along with the personalized patient pathway generator 508 is its ability to determine the ‘next best actions’ for each patient with the use of the hyper-personalized patient profiles. This is achieved by precision profiling and predictive analytics capabilities, which include various factors such as patient's response to previous treatments, evolution of their medical condition, and the efficacy of different healthcare interventions. These ‘next best actions’ are not static recommendations but dynamic suggestions that adapt as new data becomes available, thereby ensuring that the patient's care pathway is continually optimized which eventually achieves best possible outcomes through referral matching facilitated by the marketplace 208.

The integration of HL7 FHIR standards in the profile generation module 206 may ensure seamless interoperability with various EHRs and healthcare systems. This standardization may allow real-time data exchange, which is fundamental and critical in maintaining accuracy and relevance of the patient profiles. The real-time data exchange capability may also facilitate responsiveness of the profile generation module 206 to changing healthcare scenarios, allowing for the immediate incorporation of new medical data into the patient profiles and the consequent adjustment of the ‘next best actions’ by the personalized patient pathway generator 508 (also referred to as pathway generator 508 interchangeably without limitations).

The profile generation module 206 may include generative AI feedback mechanisms and generative AI and ML models together referred to as generative AI components 510 to simulate potential healthcare scenarios based on the patient's data. By analyzing these scenarios, the profile generation module 206 can refine recommendations for patient care, thereby ensuring that the ‘next best actions’ are not only based on past and present data but are also forward-looking. This may allow HDOs to prepare for potential future health challenges that patients might face.

The profile generation module 206 may include other large language models 512 to enrich and enhance granularity of the patient profiles, offering a unique depth in understanding patient needs, history, and preferences.

In embodiments, the AI and ML analysis system 504 that may include the generative AI components 510 and the other large language models 512 may allow the SEI system 200 to add a new dimension to the patient profiles, capturing nuances of the patient data, and ensuring a precision in care intervention that sets a new gold standard for care delivery.

The SEI system 200 may not work as a static system; but as an ever-evolving entity. With the generative AI components 510 and other LLMs 512 it may constantly learn, adapt, and regenerate strategies, setting a dynamic pace for healthcare interventions.

The generative AI components 510 and the other large language models 512 may help the personalized patient pathway generator 508 to forecast, simulate, or suggest a range of possible patient care pathways, enhancing the strategic aspect of interventions.

The AI and ML analysis system 504 doesn't just predict but may also create multiple healthcare pathways, enabling a dynamic, evidence-based approach to patient care.

Harnessing the collective intelligence of the other LLMs 512, the generative AI components 510, and traditional AI algorithms of the AI and ML analysis system 504, the SEI system 200 reshapes patient referral and acquisition, pushing the boundaries of what's possible in healthcare intervention.

Furthermore, the profile generation module 206 focuses on data and evidence gathering which is critical in ensuring that the patient profiles are both comprehensive and evidence-based. The profile generation module 206 may consolidate data from diverse formats, enriching it with insights derived from the generative AI components 510. This process of gathering and processing evidence may ensure that any healthcare recommendations included within the patient profiles are based on facts and any care pathways generated utilizing these profiles for the patients will likely result in best possible outcomes and best possible referrals matching through the marketplace platform 208.

The personalized patient pathway generator 508 may create an outcome indicative of the next best actions based on the hyper-personalized patient profiles. Post-auction, these pathways may guide the HDOs in delivering tailored care to patients, ensuring that each step of the treatment or care is in accordance with the individual's specific health needs and preferences. This personalization may extend beyond clinical considerations such as to include a variety of factors such as a patient's lifestyle, socio-economic context, and personal healthcare goals, without limitations.

The Strategic Evidence Intervention (SEI) system 200, through its marketplace 208, introduces a transformative approach to patient care, where clinical excellence and business optimization combine to deliver technologically equipped care plans and referrals for specialized care through best possible matchmaking. Various healthcare entities may acquire patient profiles that not only match their clinical expertise but also align with their financial objectives. For example, a specialty clinic focusing on diabetes management can identify and acquire profiles of patients who are most likely to benefit from their services, thereby ensuring that optimal patient outcomes and profitability are achieved for all participants.

By creating the hyper-personalized patient profiles and determining the ‘next best actions,’ the profile generation module 206 along with the pathway generator 208 may facilitate a healthcare delivery process that is both patient-centric and data-driven/evidence-based. This may allow that patients to receive care that is not only tailored to their individual needs but is also predictive and proactive, thereby meeting their evolving health conditions and care needs. Simultaneously, the profile generation module 206 may empower healthcare providers to make informed decisions that can optimize both clinical outcomes and business performance.

The AI and ML analysis system 504 of the profile generation module 206 may include or be coupled to the generative AI components 510 and the other large language models 512, among various other AI and ML tools and models (not shown in figure). The AI and ML analysis system 504 or the generative AI components 510 may offer a suite of generative AI models tailored to critical healthcare workflows. These advanced generative models may leverage various AI services to streamline processes and enhance operations.

The Strategic Evidence Intervention (SEI) system 200 is configured to synergize two objectives-focusing on the hyper-personalized patient profiles that best match the unique services and profit goals of healthcare delivery organizations (HDOs) and other care settings.

The SEI system 200 may be configured as an avant-garde platform designed to digest diverse health data formats, including HL7 EDI, HL7 FHIR, and other Health Information Exchange (HIE) types. The objective of the SEI system 200 is to process this data, synthesize evidence, and compute the tailored “next best actions” for each patient. This process culminates in the creation of the hyper-personalized patient profiles, pinpointing the most profitable and clinically appropriate care trajectories.

The SEI system's core benefits arise from its intricate data integration and evidence-gathering mechanisms. By consolidating and processing multifaceted health data, SEI system 200 cultivates a robust evidence base. This may be further enriched by the AI and ML analysis system 504, tasked with predicting the most suitable and profitable patient care trajectories among its various other tasks including those discussed in the document.

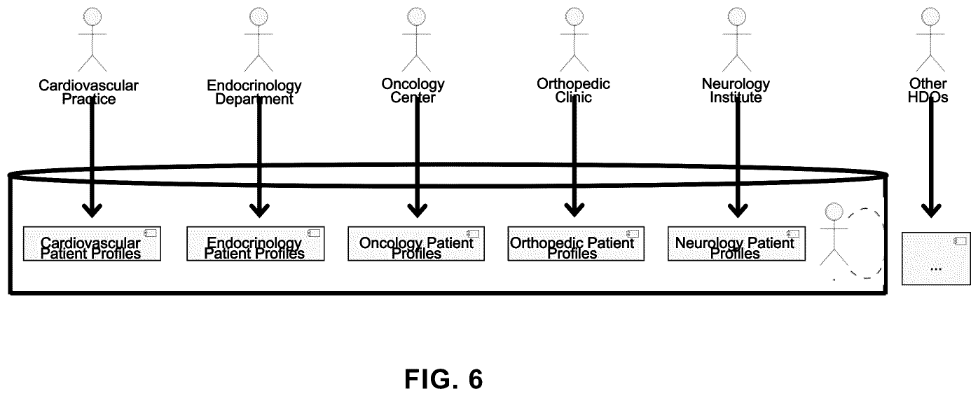

The SEI system 200 is designed to facilitate the marketplace platform 208-a specialized feature wherein the hyper-personalized patient profiles are available for acquisition. The HDOs and other care settings (like imaging centers, labs, specialty clinics) can search, evaluate, and purchase these patient profiles that align with their service offerings and profit objectives. For instance, a cardiovascular practice can query the SEI marketplace to acquire profiles where cardiovascular interventions are the “next best actions,” ensuring a profitable and clinically sound patient inflow. Similarly, an endocrinology department can target profiles indicating diabetes-related interventions. This is illustrated through a representative diagram in FIG. 6.

The SEI system 200 may be configured to equip the HDOs and other care settings with a tool to acquire the patient profiles that perfectly blend clinical needs with profitability.

The SEI system 200 may facilitate the marketplace platform 208 where various healthcare entities including the HDOs may strategically select the patient profiles, aligning them with their specific services and revenue objectives.

The SEI system 200 may allow specialty care establishments to pinpoint and acquire the most suitable patient inflow, optimizing both patient outcomes and business profitability.

The SEI system 200 may serve as a comprehensive hub where the HDOs can search, evaluate, and purchase the patient profiles, ensuring their resources are strategically utilized for the most profitable care trajectories.

The SEI system 200 implements comprehensive data security measures across all components. The data collection and integration block 202 employs encryption and anonymization engine 924 to secure incoming data streams using HIPAA-compliant encryption protocols. The processing module 204 utilizes blockchain-configured distributed access points 504 connected to the trusted ledgers system 514 to maintain data integrity through immutable audit trails. The profile generation module 206 stores sensitive patient data in encrypted containers within the non-transitory computer-readable medium 1118, with access controlled through the structured data interface 1110. The reverse auction marketplace platform 208 employs the secure transaction gateway 312 for encrypted data transmission, while the patient data privacy module 322 enforces role-based access controls. The real-time notification system 310 transmits alerts through encrypted channels, with the regulatory compliance checker 318 ensuring adherence to privacy standards. All system interactions are recorded in distributed ledgers 806 maintained across the blockchain device 802, with the private data store 818 providing secure, segregated storage for sensitive information. The feedback and optimization module 212 implements encryption protocols 1014 and access control mechanism 1016 to protect feedback data, while the pathway generator module 508 utilizes secure communication channels protected by machine learning algorithm 1006 to detect potential security anomalies. This multi-layered security architecture ensures comprehensive protection of patient data throughout its lifecycle within the system 200.

In accordance with various embodiments, the SEI system 200 may be configured to streamline processes and enhance operations in areas including the following:

(1) Prior Authorization Clinical Review: The SEI system 200 may power an automated chatbot that may quickly addresses clinicians' questions about insurance coverage for specific medical procedures or treatments. By analyzing the complex rules and regulations of various insurance plans, the SEI system 200 may provide accurate and timely information on prior authorizations. This not only reduces the time needed for manual reviews of the hyper personalized patient profiles by the HDOs but also minimizes errors, appeals, and associated costs. The result is a more efficient prior authorization process that enhances the collaboration among the participants, ultimately benefiting patient care.

(2) Medical Coding & Explanation of Benefits: The AI and ML analysis system 504 may maximize accuracy in medical coding by utilizing advanced algorithms that understand the nuances of medical terminology and billing codes. Simultaneously, the system simplifies the communication of explanation of benefits statements for patients, translating complex billing details into clear and comprehensible language. This dual approach ensures that billing is precise and transparent, reducing the likelihood of errors and disputes. Patients benefit from a clearer understanding of their financial responsibilities, while providers and payers enjoy streamlined billing processes.

(3) Referral Management: The SEI system 200 may optimize the referral process by intelligently matching patients with the right specialists based on their medical needs, insurance coverage, and geographical location. The SEI system 200 coordinates all aspects of the referral, from identifying the appropriate specialist to scheduling appointments and sharing relevant medical records. This seamless process may keep all stakeholders, including referring physicians, specialists, and patients, coordinated and informed. The result is a more effective referral system that enhances patient satisfaction, reduces wait times, and ensures continuity of care.

(4) Care Plan Creation & Management: The SEI system 200 may revolutionize care management in healthcare by automating tailored consent letters and enrollment materials, thereby serving culturally diverse populations effectively. Additionally, it streamlines workflow with real-time chatbot support and data-driven summaries, enabling case managers to focus on impactful member engagement and decision-making. This technology stands to improve efficiency and personalize care dramatically.

The embodiments herein provide various innovative features described herein and further described below, without limitations.

The SEI system 200 may provide a comprehensive platform to implement Strategic Evidence Intervention (SEI). The system 200 may integrate healthcare data sources, leverages AI and ML for analytics, and creates the hyper-personalized patient profiles. These profiles, with recommended interventions, can be auctioned in the reverse marketplace 208 for the HDOs, ensuring both superior patient care and profitability. Instead of a traditional referral network the system 200 uses a reverse auction process for patient acquisition. The SEI system, 200 offers a unique platform-as-a-service designed to streamline the process of integrating patient data, analyzing it via AI and ML algorithms, and generating the hyper-personalized patient profiles with strategic healthcare interventions.

These profiles may be made available in the reverse auction marketplace 208, enabling the Healthcare Delivery Organizations (HDOs) to acquire the profiles aligning with their expertise and profitability metrics, ensuring both optimal patient care and business success.

The concept of a reverse auction for patient acquisition, as incorporated in the SEI system 200 offers a unique opportunity in the healthcare domain.

The SEI system 200 may allow creating the hyper-personalized patient profiles and position itself as a champion of patient-centric care, where recommendations may be tailored to each patient's unique needs and circumstances.

Traditional referral systems are often based on relationships and affiliations. In contrast, the SEI system 200 based reverse auction approach ensures that the HDOs are actively competing for patients, incentivizing them to offer the best care solutions, resources, and expertise.

The reverse auction approach can promote transparency by revealing the criteria by which the HDOs bid for the patients. This transparency may bolster trust among patients and the referring providers.

By allowing the HDOs to acquire the profiles that align with their profitability metrics, the SEI system 200 may ensure that the HDOs optimize their resource utilization, ensuring both patient care and operational efficiency.

The AI and ML analytics can help in creating a truly evidence-based system, leading to better outcomes and more informed HDO bidding decisions.

With data integration and analytics, the SEI system 200 may potentially expand its scope beyond traditional referrals to areas like preventive care, chronic disease management, and post-hospitalization care, without limitations in accordance with various embodiments.

The SEI system 200 may incorporate payers and insurance providers into the reverse auction process. These participants can play a crucial role in influencing the bids and ensuring that cost-efficient and high-quality care options are selected.

The SEI system 200 may offer extensive training, demos, and educational resources to showcase the benefits of the reverse auction process to both the patients and the HDOs. By making stakeholders comfortable with this approach, adoption rates may be increased.

The SEI system 200 may ensure that the reverse auction process adheres to healthcare regulations, patient privacy, and ethical considerations. A well-regulated system can garner trust faster.

The SEI system 200 may include mechanisms in place to collect feedback from both the patients and the HDOs as has been described in the document elsewhere. This feedback can guide iterative improvements, making the system 200 even more appealing and effective.

The SEI system 200 may engage in aggressive marketing campaigns to educate the market on the benefits of the reverse auction process. Additionally, the SEI system 200 may be integrated with partner and external tools to advocate for this approach.

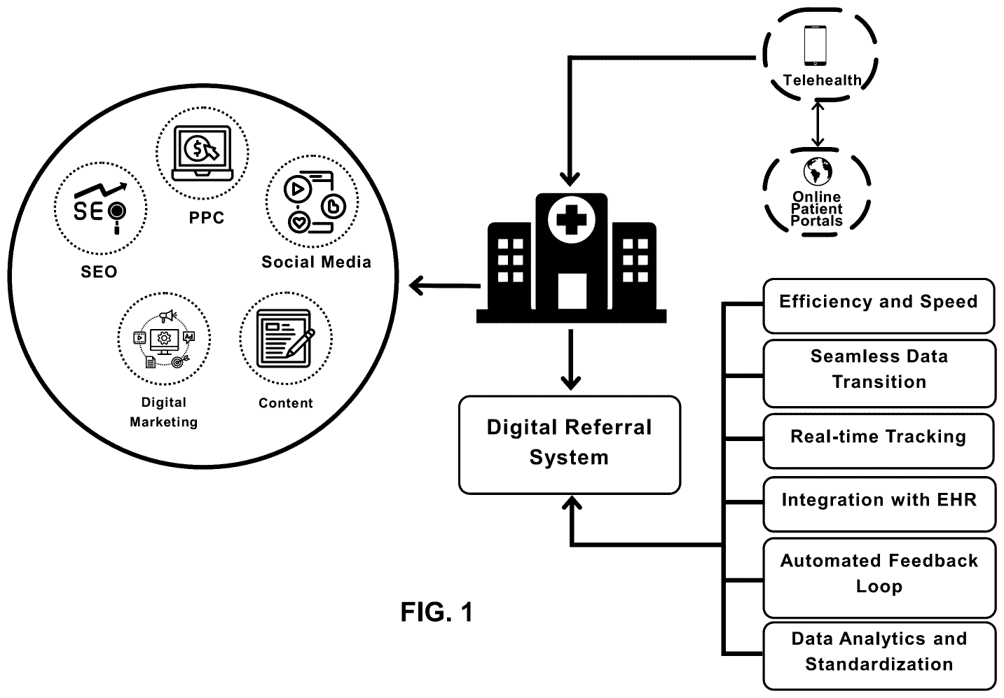

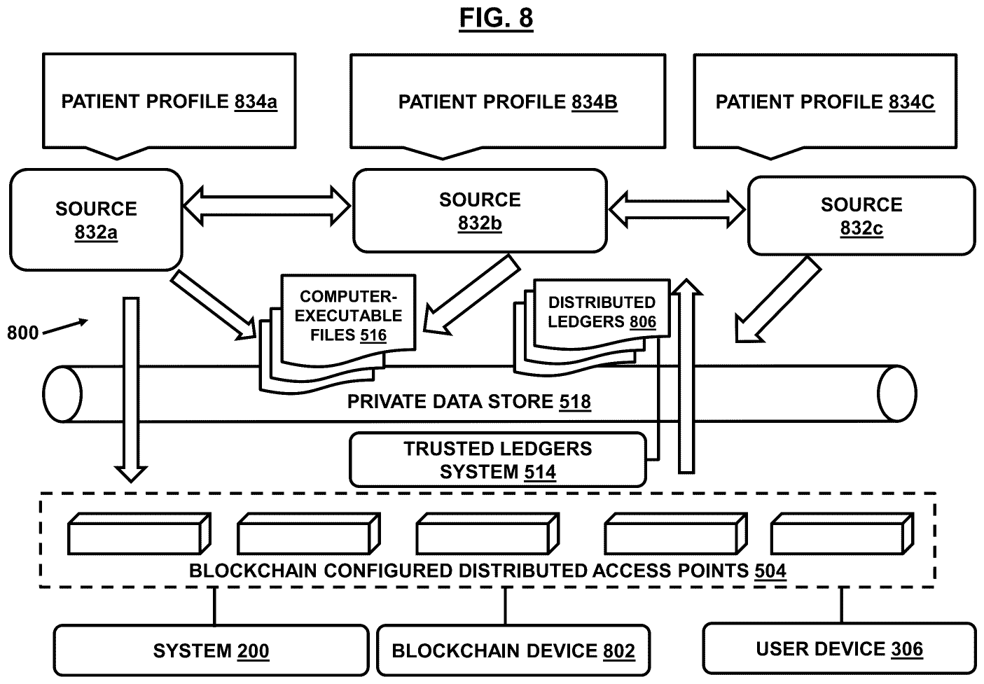

FIG. 8, with reference to FIGS. 1 through 7, illustrates an exemplary blockchain-configured ecosystem architecture 800 containing one or more components of the system 200 and also contain additional components so as to allow integrity of transactions and the digital data (including the information blocks and the precision blocks) shared/processed during the transfer or storage as discussed above in the document. The blockchain-configured ecosystem architecture 800 may provide a crowdsourced integrity network for storing the data accessed or extracted or transformed for sharing or storing across a network instead of locally stored information by different participants or databases that may be tampered with.

The ecosystem architecture 800 may be blockchain-configured involving various blockchain devices. For example, the system 200 may interact with a blockchain device 802 through a plurality of blockchain configured distributed access points 804. A network that facilitates interaction across all components may be a blockchain integrity network. The blockchain network may build trust among the various participants or entities or systems or components thereof and their associated computing terminals or devices even if the devices/terminals or machines etc. may not “know” one another. The blockchain network may allow connections and transactions and recording and sharing of the data, information blocks, precision blocks, and various codes/token generated during an entire transaction including service tokens and authorization tokens in a trusted mode. A record of transactions and sharing and data from various terminals/devices stored on the blockchain in the form of computer-executable distributed ledgers 806 may provide proof to command the necessary trust among the terminals/devices (such as those associated with various participants/nodes etc. without limitations) to cooperate through a peer-to-peer or peer-to-client distributed digital ledger technology system. The ecosystem architecture 800 may include a distributed trusted ledgers system 814 containing the distributed blockchain ledgers 806 associated with a plurality of computing terminals and devices such that each ledger stores a copy of computer-executable files 816 containing the context inputs, context patterns, precision blocks, information blocks, information extracted from the sources 832, and various other details for preparing the patient profiles such as the patient profiles 834 and the trust notes for defining security and trust among the computing terminals and devices across the network so that each computing terminal trusts the other computing terminal through the blockchain. The distributed ledgers system 814 enables coding of rules-based contracts that execute when specified conditions are met. The distributed ledgers 806 make it easier to create cost-efficient networks where any device or any evidence associated with a task execution or transaction may be tracked, without requiring a central point of control.

The various computing terminals or devices in the network serve as distributed peer-to-peer nodes and connections. The system 200 and its components thereof may be configured to perform the task of processing the context inputs and the information blocks further through the blockchain network based on the rules as defined and discussed herein. Each terminal/device/node in the ecosystem architecture 800, etc. may receive a copy of the blockchain which may get downloaded automatically upon joining the blockchain integrity network. Every permissioned node or the device in the network is an administrator of the blockchain, and may join the network voluntarily so that the network is decentralized.

The blockchain may eliminate the risks that come with data being held centrally by storing data across the network which may include the computer-executable files 816 containing the information blocks, context inputs, context patterns, etc. and/or the various tokens/codes including transaction codes. The blockchain security use encryption technology and validation mechanisms for security and integrity verification. The security may be enabled through public and private keys. A public key may define a user's address on the blockchain. The private key may give its owner an access to various digital assets in the network.

In an embodiment, the distributed ledgers 806 may enable coding of smart contracts (with the use of such as smart contract systems) that will execute when specified conditions are met. These smart contracts may protect various information pieces associated with the service deliveries and other transactions and data processing/storage and eliminate the risk of files copying and redistribution without protecting privacy rights.

The blockchain-configured ecosystem architecture 800 may provide a private view for the various devices and the entities operating in the network through the private data store 618 so that each permissioned device such as the system 200 may privately access the computer-executable files 816 associated with a task or user inputs and requests for content consumption based on various policies such as based on their respective identities. The system 200 may access the computer-executable files 816 through the dedicated private data store 818 available through the plurality of distributed blockchain-configured access points 804, which may be enabled in the form of distributed blocks as shown in FIG. 8, with each block providing the ability to access the features of the blockchain-configured ecosystem architecture 800 by different terminals and devices at the same time based on defined and granted access rights.

The private data store 818 may provide a virtual storage to facilitate interaction, information exchange, reviewing, and presentation of the computer-executable files 816. For example, the private data store 818 may allow a virtual storage and presentation of only limited executable files or portions of the executable files for access by particular entities or participants in accordance with permissions granted for reviewing. The private data store 818 may be configured to auto-hash review interactions at any required interval. This compartmentalization of the computer-executable files 816 ensures that the computer-executable files 816 are secured and private as per access rights authorized to the nodes. The data presented on the private data store 818 of the blockchain serves as a secure way to ensure that the private data store 818 is in sync with any permissioned access.

In an embodiment, the blockchain-configured digital ecosystem architecture 800 may provide a federated blockchain comprising of several entities/participants (including the user) and their associated computers and devices and sensors that jointly interact to process transfers of data through a trusted, secured and distributed network of the blockchain-configured access points 804.

The SEI System 200 is an advanced system utilizing artificial intelligence (AI) and machine learning (ML) technologies, specifically configured for the optimization of patient referral management within a digital healthcare marketplace. The system 200 may leverage a sophisticated AI algorithm designed to analyze various data points, including but not limited to, patient medical histories, provider specialties, geographic locations, and provider availability. Machine learning components of the SEI System 200 may be structured to continually learn from incoming data, thereby enhancing the accuracy and relevance of its referral suggestions over time.

Some of the applications of the SEI system 200 are listed below in various embodiments, without limitations.

Enhanced Interoperability Utilizing FHIR Standards: This embodiment may involve utilization of Fast Healthcare Interoperability Resources (FHIR) standards to enable advanced interoperability with existing Electronic Health Records (EHRs) and other healthcare systems. It may facilitate real-time data exchange, significantly reducing patient wait times through instantaneous referral processing.

AI-Driven Provider Matching System: The embodiments herein may include an AI-powered algorithm that matches patients with providers. The system 200 considers individual medical needs, provider expertise, patient preferences, and historical patient outcomes to optimize the match. The system 200 may employ AI to assess the urgency of referrals, enhancing the efficiency of patient care.

Automated Prior Authorization Process: This aspect of the embodiments herein may leverage AI to streamline the prior authorization process. The system 200 may include predictive functionalities to identify referrals requiring prior authorization and automate the completion of necessary details, thereby expediting the referral process. Intuitive User Interface Design: The system 200 may include a responsive, user-friendly interface adaptable to various devices. The interface may feature a customizable dashboard for the providers (HDOs) to efficiently manage referrals, including viewing and tracking pending, accepted, and completed referrals.

Predictive Analytics for Workflow Optimization: This embodiment may utilize the AI for predictive analytics, foreseeing bottlenecks or peak times in the referral system 200. This may allow for the optimization of workflows and resources, and provide insights into potential health outcomes influenced by referral patterns.

Enhanced Communication Tools: The system 200 may include integrated communication tools such as chat, video, and voice capabilities for immediate consultation between healthcare providers. The system 200 may support secure messaging between patients and providers within the platform.

AI-Enabled Smart Scheduling Feature: The system may implement the AI tools for scheduling, considering factors like provider availability, patient preferences, and urgency to suggest optimal appointment times.

Feedback Loop Optimization: The system 200 may enhance feedback loop by using AI to summarize key information from referral outcomes for the referring provider. The system 200 may automate the feedback process, keeping providers updated in real-time.

Data-Driven Continuous Learning: Machine learning algorithms may be employed to continuously improve the referral process, adapting based on observed patterns, feedback, and outcomes.

Blockchain-Based Data Security: Blockchain technology may be utilized for secure, tamper-proof record-keeping, thereby ensuring data integrity and trust in the referral process.

Integrated Telehealth Functionalities: The system 200 may integrate telehealth capabilities to facilitate virtual consultations, enhancing the flexibility and reach of healthcare services.

Patient Portal with AI Assistance: A patient portal may be provided, allowing patients to track their referrals, communicate concerns, and receive AI-driven advice or responses to common queries.

Performance Benchmarking Tools: Analytics tools may be included for benchmarking performance against industry standards, aiding healthcare organizations in identifying and addressing areas for improvement, in some embodiments.

Customizable and Scalable Platform Design: The system 200 may be designed to be customizable to the specific needs of various healthcare organizations (HDOs) and scalable to accommodate their growth in accordance with some embodiments herein.

Integration with Wearables and IoT Devices: The system 200 may allow for the integration of data from wearable health technologies and IoT devices, providing a more comprehensive view of patient health and informing referral decisions.

The system 200 may be configured to address critical pain points in current digital referral processes by prioritizing a seamless user experience, leveraging advanced analytics, and ensuring comprehensive interoperability. The design of the system 200 may include features and capabilities to address the needs of all stakeholders in the healthcare ecosystem, from physicians to patients, ensuring that it not only meets but exceeds their expectations in delivering efficient, personalized healthcare services, in accordance with various embodiments as discussed herein in this document without limitations.

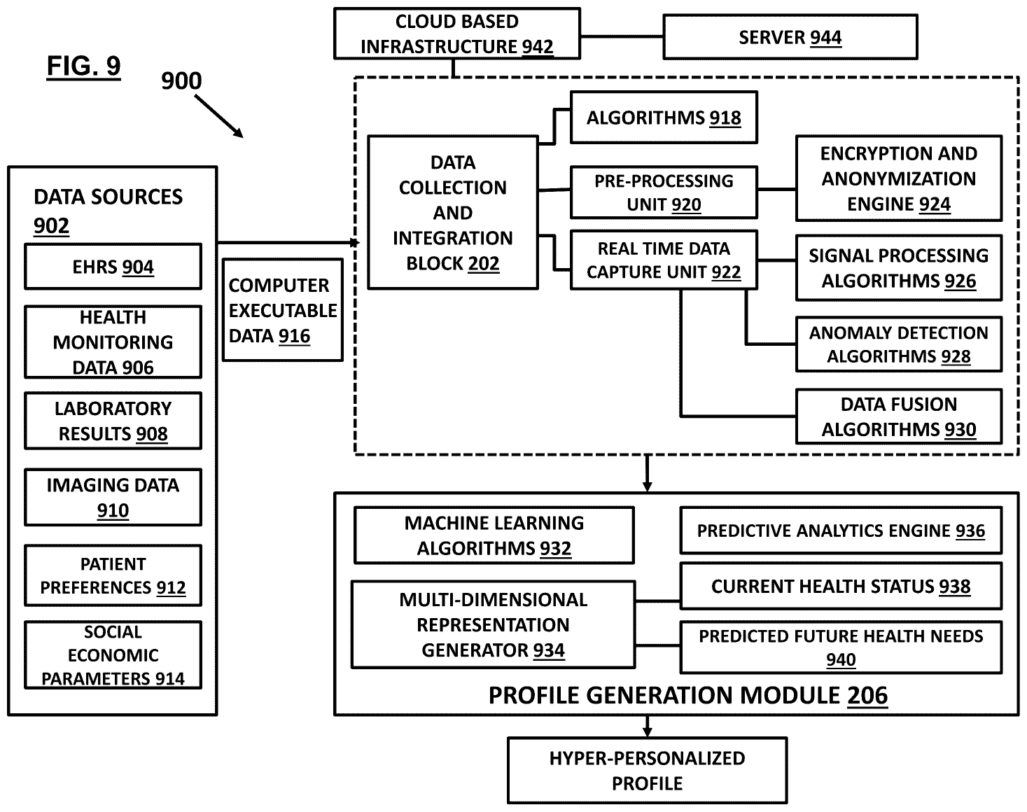

FIG. 9 illustrates a profile generation module 206 configured to generate the hyper-personalized profile of a subject based on various data sources 902, in accordance with an embodiment. In various embodiments, the SEI system 200 is configured to facilitate generating one or more hyper-personalized care pathways or next best actions for the subject. The system 200 may leverage the data sources 902 and advanced computational techniques, including such as machine learning algorithms 932 and predictive analytics, to provide the personalized care pathways. The system 200 is designed to dynamically adapt and respond to the current health status 938 of the subject, as well as predict future healthcare needs (represented by predicted future health needs 940 in the FIG. 9) based on real-time and historical data.

As discussed above, the system 200 may include the data collection and integration module (also referred to as data collection and integration block 202), which serves as an initial interface for receiving and processing a range of computer executable data 916 from the disparate data sources 902. These data sources 902 may include structured data sources, semi-structured data sources, and unstructured data sources such as but are not limited to, electronic health records (EHRs) 904, real-time health monitoring data 906, laboratory results 908, imaging data 910, patient preferences 912, and socio-economic parameters (also referred to as social-economic parameters 914). The data integration module 202 may be operably connected to various other components of the SEI system 200, facilitating seamless data flow from the data sources 902 into system architecture.

The EHRs 904 may include a wide array of longitudinal patient data, including medical history, diagnoses, treatment history, medication prescriptions, and other relevant clinical information. The EHRs 904 may provide foundational medical data necessary for understanding a healthcare journey of the subject and offer critical insights into previous interventions, therapeutic responses, and potential comorbidities. The real-time health monitoring data 906, which may be collected from wearable devices or other sensor technologies, may provide continuous or near-continuous information on vital signs such as heart rate, blood pressure, blood glucose levels, oxygen saturation, and physical activity, and the like without limitations. An inclusion of this data helps in real-time assessments of the subject's health and facilitates detection of emergent health concerns or medical events that may require immediate intervention in relation to the subject.

The laboratory results 908 may include diagnostic tests, blood work, imaging results, and pathology reports without limitations. This kind of data allows for identification of biomarkers, disease markers, and other key physiological indicators that may be essential for tracking disease progression or response to treatment about the subject. The data sources 902 may include the imaging data 910, which may consist of medical scans such as MRIs, CT scans, and X-rays, that assists in the assessment of anatomical structures, disease states, and potential complications. These data sources 902, when integrated into the SEI system 200, allows for a comprehensive understanding of the health of the subject, which facilitates in creation the personalized care pathway that may incorporate not only physiological and biochemical data but also visual representations of anatomy of the subject.

In accordance with various embodiments, in addition to these traditional medical data types, the SEI system 200 may ingest data relating to the patient's preferences (also referred to as patient preferences 912) and socio-economic parameters 914. The patient preferences 912 help in personalizing the care pathways, as they reflect the subject's values, goals, and desires in terms of their healthcare needs. The patient preferences 912 may include treatment options, lifestyle choices, and desired outcomes. The integration of socio-economic data further enriches the personalized pathways by considering external factors such as the subject's access to healthcare services, financial constraints, social support systems, and other factors that may influence the feasibility and effectiveness of certain healthcare interventions and accordingly need a different care pathway next best actions. Throughout this document, the next best actions may be used to refer to the care pathways or the next best actions may be used in a way such that a care pathway may include multiple next best actions.

The data integration module 202 may be configured to process and standardize these heterogeneous data sources 902 into a consistent, machine-readable format suitable for subsequent analysis by various components of the SEI system 200. The data integration module 202 may include functionality for data cleaning, transformation, and normalization. In embodiments, the data integration module 202 may be capable of handling data privacy and security considerations, such that sensitive computer executable health information (also referred to as computer executable data 916) is protected in compliance with relevant regulatory standards such as HIPAA (Health Insurance Portability and Accountability Act) and GDPR (General Data Protection Regulation).

The data collection and integration block 202 may include or be coupled to a pre-processing unit 920. The pre-processing unit 920 may be configured to handle and process raw, heterogeneous data originating from the data sources 902, including but not limited to the electronic health records (EHRs) 904, real-time health monitoring data 906, laboratory results 908, imaging data 910, patient preferences 912, and the socio-economic parameters 914. The pre-processing unit 920 may be responsible for transforming these disparate data types into a consistent, uniform format suitable for further analysis by downstream modules such as the profile generation module 206.

The pre-processing unit 920 may perform data cleaning, which may be executed by applying algorithms 918 designed to detect and correct errors or inconsistencies within the raw data. These algorithms 918 may identify missing, duplicate, or erroneous entries, and employ data imputation techniques based on historical trends or contextual analysis to resolve these issues. In embodiments where data points cannot be meaningfully imputed, they may be flagged for further review or removed from consideration to prevent data integrity issues.

The pre-processing unit 920 may also perform data normalization. Given that the SEI system 200 ingests data from the various data sources 902, each employing potentially different units or scales, the pre-processing unit 920 may standardize measurements into a common reference frame. For example, heart rate data may be received in beats per minute (BPM) from wearables, while blood pressure data may be presented in millimeters of mercury (mmHg). The pre-processing unit 920 may apply conversion algorithms 918 to ensure that all health parameters are represented in consistent, universally accepted units, facilitating cross-referencing and comparison.

The pre-processing unit 920 may be equipped to handle complex data integration tasks. Specifically, the pre-processing unit 920 may be configured to consolidate temporal and spatial data from multiple modalities such as time series data from real-time sensors and categorical data from EHRs 904, by applying advanced alignment techniques. These techniques allow that data points are synchronized in time, even when captured at varying frequencies. For example, data from continuous glucose monitors may be aligned with daily records from laboratory blood tests to create a coherent health timeline.

The pre-processing unit 920 may include an encryption and anonymization engine 924 that is configured to encrypt and anonymize the data in accordance with compliance of regulatory frameworks, such as HIPAA and GDPR, regarding data privacy and security. The encryption and anonymization engine 924 applies cryptographic methods to anonymize sensitive patient data before it enters the processing pipeline, such that unauthorized access is prevented throughout the data handling process.

The data collection and integration block 202 may further include a real-time data capture unit 922 that is configured to continuously receive, monitor, and process live health data from the data sources 902 such as sensors, wearables, and other monitoring devices. The real-time data capture unit 922 provides dynamic, up-to-date health assessments for the subject.

The real-time data capture unit 922 interfaces directly with devices such as continuous glucose monitors (CGMs), heart rate sensors, blood pressure cuffs, electrocardiograms (ECGs), and activity trackers, among others without limitations. These devices may generate continuous streams of health data, which may then be transmitted to the real-time data capture unit 922 for immediate processing. The real-time data capture unit 922 may be capable of handling a high throughput of data and is designed to efficiently filter and parse raw data to extract relevant health metrics.

The real-time data capture unit 922 may utilize advanced signal processing algorithms 926 to filter out noise and correct errors in sensor data. For example, real-time data capture unit 922 employs frequency-domain analysis to distinguish between actual physiological signals and environmental noise, to ensure that only valid, clinically relevant data is processed. The real-time data capture unit 922 incorporates anomaly detection algorithms 928, which are programmed to identify outlier values that fall outside the normal physiological range. Such anomalies may include such as a sudden spike in heart rate or blood glucose levels that may trigger an immediate alert within the SEI system 200, notifying both the healthcare provider and the subject for prompt intervention.

In some embodiments, the SEI system 200 may integrate the real-time data capture unit 922 with a cloud-based infrastructure 942 for data transmission across networks. The SEI system may use wireless protocols such as Bluetooth, Wi-Fi, or 5G for transmitting the data from the real-time data capture unit 922 with low latency, such that near-instantaneous updates to the system's health profile are maintained. This low-latency ability of the SEI system 200 may be critical in scenarios requiring immediate intervention, such as acute health events or sudden deteriorations in the subject's condition. A high-bandwidth communication channel may be used by the real-time data capture unit 922 for handling large volumes of real-time data, including high-resolution imaging or continuous biometric measurements, that may be transmitted efficiently to a central server.

The real-time data capture unit 922 applies real-time data fusion algorithms 930 to integrate sensor data into a unified health model. For example, the physiological data such as heart rate, blood pressure, and blood oxygen levels are dynamically incorporated to create a real-time snapshot of the subject's health status. These fused data points may then be used by machine learning models to update the personalized health profile of the subject in real time.

The real-time data capture unit 922 is configured to support remote patient monitoring (RPM) and telemedicine applications. The data captured from the real-time data capture unit 922 can be securely transmitted to healthcare providers for remote evaluation, allowing clinicians to make timely decisions without requiring the subject to be physically present. The real-time data capture unit 922 transmits the data in compliance with relevant privacy regulations, using secure data protocols such as Transport Layer Security (TLS) and secure sockets layer (SSL) encryption. The real-time data capture unit 922 is configured to operate autonomously and continuously, such that the system 200 always has access to the most current health information.

The processed data is transmitted to the profile generation module 206. The profile generation module 206 is operatively coupled to the data collection and integration block 202 and is responsible for synthesizing the ingested data into the hyper-personalized profile of the subject. The profile generation module 206 includes one or more machine learning algorithms 932 and a predictive analytics engine 936, and a multi-dimensional representation generator 934 to generate a multi-dimensional representation of the subject's current health status 938 as well as predicted future healthcare needs 940.

The machine learning algorithms 932 are trained on large datasets that may include historical health data, clinical trial data, and real-world evidence, allowing the profile generation module 206 to identify complex patterns and relationships between various health factors. The machine learning algorithms 932 can utilise both supervised and unsupervised learning techniques, such as decision trees, neural networks, support vector machines, and clustering algorithms, to generate meaningful insights from the data. The machine learning algorithms 932 may be further refined using reinforcement learning models, where the system 200 continuously learns and adapts based on feedback from the subject's evolving health data.

The synthesis of data through these machine learning algorithms 932 allows for the creation of the hyper-personalized computer executable profile alternatively referred to as profile for simplicity purposes) that captures not only the subject's present health status but also accounts for their historical health data, treatment responses, and demographic information. The profile serves as a dynamic, real-time representation of the subject's health, providing insights for healthcare providers and other stakeholders involved in the care process.

The multi-dimensional profile may include categories such as current health status 938. This dimension provides an up-to-date view of the subject's health, including biomarkers, lab results, vital signs, and other indicators. It provides a snapshot of the subject's physiological condition at any given time. The multi-dimensional profile may include categories such as historical health data. This dimension captures the longitudinal health history of the subject, incorporating past diagnoses, treatments, medications, and outcomes. It allows the system 200 to identify trends and changes in the subject's health over time. The multi-dimensional profile may include categories such as predicted future healthcare needs 940. This dimension utilizes the predictive analytics engine 936 to forecast the subject's future health trajectory, based on historical data and machine learning models. The system 200 may predict an onset of new health conditions, disease progression, or the potential need for interventions such as hospitalization, surgeries, or changes in medication based on the computer executable hyper-personalized profile.

The multi-dimensional profile may further include personalized care preferences. This dimension includes the subject's expressed preferences and values, so that recommended care pathway aligns with the subject's goals and lifestyle choices. The multi-dimensional profile may include socio-economic considerations. This dimension integrates socio-economic data, including access to healthcare, financial constraints, and social support, which may be essential for personalizing the care pathway or the next best actions to the subject's specific circumstances. The hyper-personalized profile is not static but evolves continuously as new data is ingested and as the subject's health status changes. The dynamic nature of the profile allows the SEI system 200 to provide real-time updates and recommendations, so that healthcare providers can respond promptly to changes in the subject's condition.

The machine learning algorithms 932 and the predictive analytics engine 936 facilitates profile generation. In some embodiments, the predictive analytics engine 936 may utilize the data gathered from the subject to forecast potential future health events, including such as disease progression, complications, and the likelihood of needing specific treatments or interventions without limitations. The predictive analytics engine 936 may include predictive models that can account for a variety of factors, including genetic predispositions, environmental influences, lifestyle choices, and previous treatment responses.

The machine learning models may be trained to recognize patterns in the data that may not be immediately apparent to human clinicians. For example, the profile generation module 206 may detect subtle changes in lab results or vital signs that indicate the onset of a medical condition before it becomes clinically apparent. The predictive capability of the system 200 allows early intervention, which improves patient outcomes, particularly in chronic disease management and preventative healthcare.

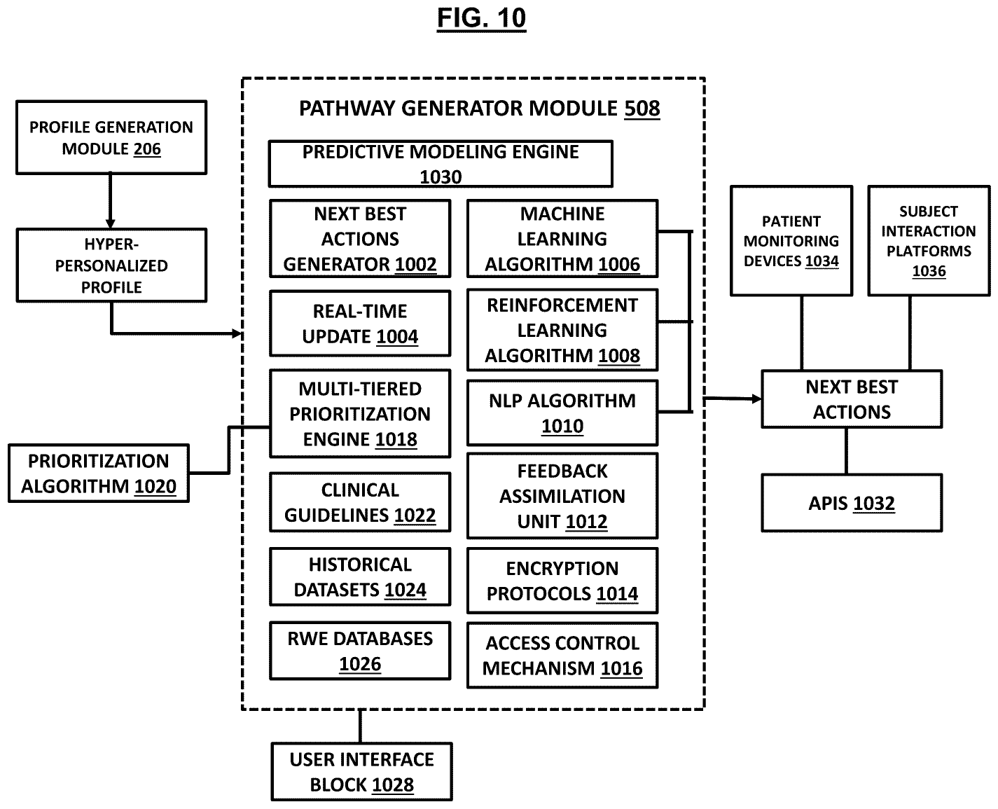

In accordance with the present disclosure, FIG. 10 illustrates a detailed representation of a decision-support architecture for care delivery integrated within the SEI system 200. The architecture includes the pathway generator module 508 that is configured to generate hyper-personalized recommendations based on the multi-dimensional profile of the subject, as synthesized by the profile generation module 206. The pathway generator module 508 is operably coupled to both the profile generation module 206 and a user interface block 1028, providing a transfer of computationally derived recommendations to end-users, including healthcare providers and the subject or devices associated with the subject and the healthcare providers.

The pathway generator module 508 includes a next best actions generator 1002, also referred to alternatively as a recommendation engine, a feedback assimilation unit 1012, and a multi-tiered prioritization engine 1018. The next best actions generator 1002 is configured to analyze the hyper-personalized profile of the subject in conjunction with a plurality of clinical guidelines 1022, historical treatment efficacy datasets (also referred to as historical datasets 1024), and real-world evidence databases (also referred to as RWE databases 1026). In particular, the recommendation generation engine may leverage machine learning algorithms 932, such as supervised learning models and reinforcement learning frameworks (also referred to as reinforcement learning algorithm 1008), to dynamically generate the next-best-actions tailored to the hyper-personalised profile of the subject.

The next-best-actions generator 1002 may be further equipped to account for temporal factors and treatment timelines by utilizing predictive modelling techniques from a predictive modelling engine 1030 coupled to the next best actions generator 1002. For example, the next-best-actions generator 1002 may predict an optimal timing for an intervention or a next best action based on the progression of the subject's condition as indicated by historical health data and real-time metrics contained in the profile. In embodiments, the next-best-actions generator 1002 applies a scoring algorithm to rank potential actions based on parameters such as efficacy, feasibility, risk factors, and alignment with the subject's preferences and socio-economic conditions.

The feedback assimilation unit 1012 is configured to receive input data from various stakeholders, including clinicians, caregivers, and the subject. The feedback assimilation unit 1012 is operatively linked to the user interface block 1028, for receiving real-time updates 1004 based on user-provided feedback regarding feasibility, acceptability, and observed outcomes of previously recommended next best actions. The feedback assimilation unit 1012 may be coupled to the machine learning algorithms 932 and its reinforcement learning mechanism (also referred to as reinforcement learning algorithm 1008) to iteratively refine the next best actions generated by the next-best-actions generator 1002.

The multi-tiered prioritization engine 1018 may be configured to stratify the generated next best actions into actionable categories, such as immediate, short-term, and long-term interventions. The stratification may be achieved through a prioritization algorithm 1020 that may evaluate urgency and criticality of each recommended action in light of the subject's current health status 938 and predictive analytics outcomes. For example, in scenarios where the SEI system 200 identifies a high likelihood of an acute medical event, the multi-tiered prioritization engine 1018 may assign a corresponding intervention a higher priority and ensures its immediate communication to the healthcare provider through the user interface block 1028.

In some embodiments, the next-best-actions generator 1002 is configured to operate in a closed-loop manner, wherein the execution of a next best action triggers an automated update to the hyper-personalized profile of the subject. This update may be facilitated by a real-time update system and a subsequent recalibration of the next-best-actions generator 1002. The closed-loop operation allows that the SEI system maintains a dynamic and responsive framework for healthcare decision-making.

The pathway generator module 508 may also include a natural language processing algorithm (also referred to as NLP algorithm 1010) to transform complex computational outputs into user-friendly explanations. For example, the pathway generator module 508 may detail how a specific next best action or recommendation aligns with established clinical guidelines 1022 or cite relevant data patterns observed in the subject's historical and real-time health records.

The pathway generator module 508 integrates robust data security and compliance measures to ensure that sensitive health information is handled in accordance with regulatory frameworks such as HIPAA and GDPR. The pathway generator module 508 includes encryption protocols 1014 and access control mechanisms (also referred to as access control mechanism 1016) to prevent unauthorized access and ensure the confidentiality of the subject's health data throughout the recommendation generation and communication process.

In certain embodiments, the next-best-actions generator 1002 is configured to support interoperability with external healthcare systems and databases. The interoperability framework enables the module to access additional datasets, such as drug interaction databases, clinical trial repositories, and pharmacogenomics libraries, further enhancing the precision and reliability of the generated recommendations.

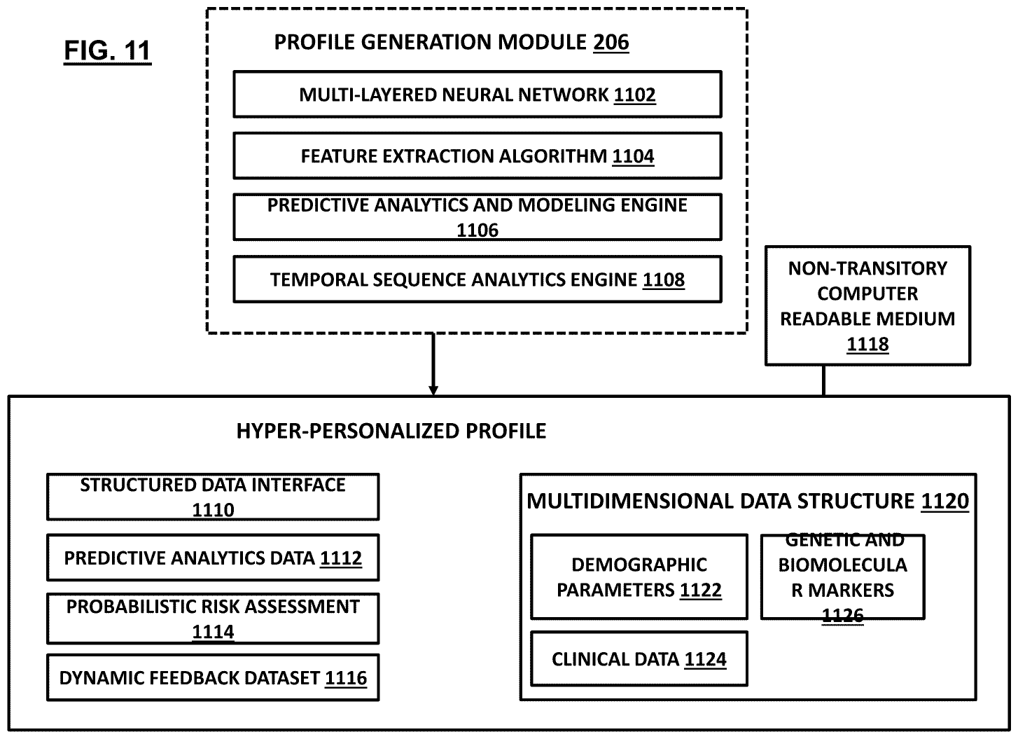

FIG. 11 illustrates the profile generation module 206 generating the hyper-personalized profile, in accordance with some embodiments. It must be understood that only additional components of the profile generation module 206 are illustrated in the figure. The profile generation module 206 may further include one or more components such as those show in previous FIGS. 1-10, without limitations.

In accordance with various embodiments, the profile generation module 206 is operatively coupled to the data collection and integration block 202 and is configured to process the synthesized computer-executable data derived from the disparate data sources 902 to generate the hyper-personalized computer-executable profile of the subject. The profile generation module 206 may comprise a multi-layered neural network 1102, a feature extraction algorithm 1104, a predictive analytics and modelling engine 1106, and a temporal sequence analytics engine 1108, each operable to perform distinct but interrelated functions for the processing and synthesis of the data.

The multi-layered neural network 1102 included in the profile generation module 206 may be specifically configured to analyze the synthesized computer-executable data. The multi-layered neural network 1102 comprises multiple interconnected layers, including but not limited to input layers, hidden layers, and output layers, each designed to process and transform the data through non-linear activation functions. The input layer may receive the synthesized computer-executable data, which may include structured, semi-structured, and unstructured data derived from the data sources 902 such as discussed above. In embodiments, the hidden layers of the neural network may employ feature extraction techniques using the feature extraction algorithm 1104 to identify one or more latent variables from the input data. The latent variables may be indicative of clinical parameters, such as physiological biomarkers, disease markers, or treatment efficacy metrics, and non-clinical parameters, such as socio-economic constraints, lifestyle preferences, and behavioral patterns of the subject. The feature extraction process may be facilitated by an application of advanced computational methods, including convolutional operations for imaging data 910, recurrent connections for sequential data, and attention mechanisms for heterogeneous data integration.

The feature extraction algorithm 1104 embedded within the multi-layered neural network 1102 is designed to autonomously identify complex patterns and relationships within the data. For example, the feature extraction algorithm 1104 may isolate significant predictors of health outcomes, such as variations in blood glucose levels, trends in medication adherence, or the presence of comorbid conditions, by analyzing historical and real-time datasets in combination. The feature extraction algorithm 1104 ensures that only the most relevant variables are selected for downstream processing, thereby reducing computational complexity and improving accuracy of the hyper-personalized profile associated with the subject.

The predictive analytics and modelling engine 1106, as part of the profile generation module 206, is configured to utilize the latent variables identified by the multi-layered neural network 1102. The predictive analytics and modelling engine 1106 may employ advanced machine learning algorithms 932, such as ensemble methods, decision trees, gradient boosting, and support vector machines, to forecast potential health trajectories of the subject. The predictive analytics and modelling engine 1106 may also incorporate probabilistic models, such as Bayesian networks, to account for uncertainties and variabilities in the input data. These predictions generated by the predictive analytics and modelling engine 1106 may be integral to the hyper-personalized profile, providing insights into potential disease progression, therapeutic efficacy, and preventative care strategies.

The temporal sequence analytics engine 1108 of the profile generation module 206 is configured to analyze time-series data embedded within the synthesized computer-executable data. The temporal sequence analytics engine 1108 is designed to process sequential and temporal information, such as changes in vital signs, fluctuations in laboratory parameters, and trends in health monitoring data 906 over time. The temporal sequence analytics engine 1108 employs techniques such as long short-term memory (LSTM) networks, gated recurrent units (GRUs), and dynamic time warping (DTW) to identify patterns and correlations across temporal datasets. The predictive analytics and modeling engine 1106 may include the temporal sequence analytics to anticipate a change in the current health status 938 of the subject. The predictive analytics and modeling engine 1106 may be trained on a plurality of historical datasets 1024 associated with the subject to refine the hyper-personalized computer executable profile iteratively.

In embodiments, the temporal sequence analytics engine 1108 ensures that the temporal dynamics of the subject's health data are accurately represented within the hyper-personalized profile. For example, the temporal sequence analytics engine 1108 may detect recurring patterns indicative of seasonal variations in health conditions, such as exacerbations of respiratory diseases during certain months, or it may identify anomalies in longitudinal health data, such as a sudden deterioration in kidney function markers, which may necessitate immediate clinical attention.

The combined functionality of the multi-layered neural network 1102, feature extraction algorithm 1104, predictive analytics and modelling engine 1106, and the temporal sequence analytics engine 1108 facilitates the creation of the hyper-personalized profile of the subject. The hyper-personalized profile is a multi-dimensional representation of the subject's health status, dynamically updated as new data is ingested by the system 200. The profile generation module 206 ensures that the hyper-personalized profile is not only a comprehensive reflection of the subject's current health state but also a predictive tool for identifying potential future health risks and informing the next-best actions. The hyper-personalized profile is computer-executable and may serve as a foundational element for driving the personalized care pathways and clinical decision-making processes.

In certain embodiments, the hyper-personalized computer-executable profile comprises a multidimensional data structure 1120 stored in a non-transitory computer-readable medium 1118. The multidimensional data structure 1120 may be configured to integrate one or more data components, including demographic parameters 1122 mapped to healthcare utilization models, clinical data 1124 encoded in accordance with HL7 FHIR schema for standardization and interoperability, and genetic and biomolecular markers 1126 processed through feature extraction algorithms (also referred to as feature extraction algorithm 1104).

The demographic parameters 1122 included within the multidimensional data structure 1120 may comprise attributes such as age, gender, ethnicity, socioeconomic status, geographical location, and occupation. These parameters may be processed and mapped to the healthcare utilization models using statistical and computational methodologies. The mapping may involve employing machine-learning-based clustering algorithms, including but not limited to k-means or Gaussian Mixture Models, to identify and predict patterns of healthcare resource consumption specific to various demographic cohorts. The processing may allow the SEI system to establish correlations between demographic attributes and healthcare utilization trends, which are stored in the multidimensional data structure 1120. The clinical data 1124 integrated into the multidimensional data structure 1120 may be encoded using the HL7 FHIR schema to ensure standardization and interoperability with external healthcare systems. The encoded clinical data 1124 may include, but is not limited to, observations, diagnostic results, medication records, and treatment plans. The genetic and biomolecular markers 1126 incorporated into the multidimensional data structure 1120 may be processed through the feature extraction algorithm 1104. These markers may include single nucleotide polymorphisms (SNPs), gene expression profiles, and proteomic data without limitations, which may be subjected to dimensionality reduction techniques, such as principal component analysis (PCA) or autoencoders, to identify latent features indicative of biological patterns. The processed genetic and biomolecular markers 1126 may then be encoded and integrated into the multidimensional data structure 1120 alongside the demographic and clinical data 1124 components.

In further embodiments, the hyper-personalized computer-executable profile comprises predictive analytics data 1112 derived from a probabilistic risk assessment 1114. The probabilistic risk assessment 1114 is performed using gradient-boosted decision trees (GBDT), trained on a multi-modal dataset that includes clinical, demographic, and genetic data. The GBDT may employ iterative boosting techniques to minimize prediction errors and improve accuracy. The probabilistic risk assessment 1114 quantifies the likelihood of future medical events, including disease onset, adverse reactions, or hospitalization, and incorporates confidence intervals, feature importance scores, and decision thresholds. This predictive analytics data 1112 may be stored as part of the hyper-personalized profile, enabling healthcare providers to access probabilistic insights for pre-emptive intervention strategies.

The hyper-personalized computer-executable profile may also include a dynamic feedback data set 1116, which reflects real-time adjustments to the next-best actions based on subject monitoring data and prior intervention outcomes. The dynamic feedback data set 1116 is generated through a reinforcement learning framework 1008, such as Q-learning or deep deterministic policy gradients (DDPG), configured to optimize healthcare recommendations over time. For example, real-time glucose monitoring data from wearable devices may be ingested into the SEI system, and based on the subject's historical response to insulin dosage adjustments, the SEI system may recalibrate future dosage recommendations. The dynamic feedback data set 1116 maintains a temporal sequence of interventions, corresponding responses, and resultant health outcomes, enabling continuous system learning and optimization of healthcare delivery.

The hyper-personalized computer-executable profile comprises a structured data interface 1110 configured to facilitate extraction of healthcare insights and integration with external healthcare delivery systems. The structured data interface 1110 may include machine-interpretable APIs 1032 that expose the data structure in standardized formats, such as JSON or XML, and implement RESTful communication protocols. The structured data interface 1110 may support queries for specific healthcare insights, such as disease risk scores or treatment recommendations, and facilitates bidirectional data exchange with the external systems, including electronic health record platforms and clinical decision support systems. For example, an external system may query the structured data interface 1110 for a subject's predictive analytics data 1112 or contribute updated clinical observations, which are dynamically incorporated into the hyper-personalized profile to ensure up-to-date and comprehensive healthcare insights.

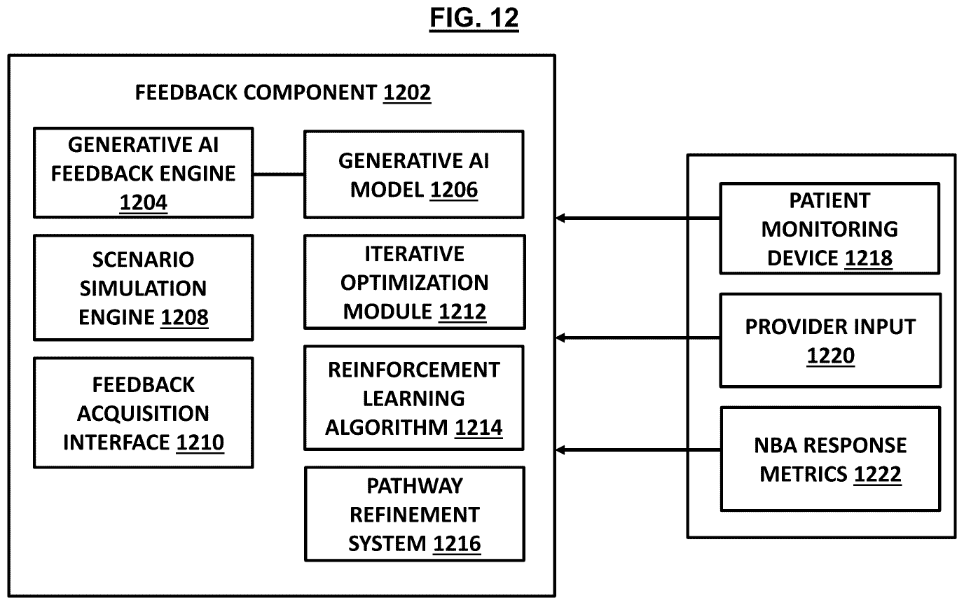

Referring to FIG. 12, an exemplary embodiment of a feedback component 1202 that may be operatively and communicatively coupled to the pathway generator module 508 and the hyper-personalized computer-executable profile. The feedback component 1202 is configured to refine subsequent recommendations or next best actions generated by the pathway generator module 508 by incorporating outcomes of the next-best actions into the hyper-personalized profile. This iterative feedback mechanism facilitates in achieving dynamic adaptability and improved accuracy in personalized healthcare delivery.

The feedback component 1202 includes a generative AI feedback engine 1204, which further comprises a scenario simulation engine 1208. The generative AI feedback engine 1204 is operatively connected to the hyper-personalized profile and leverages domain-specific computer-executable datasets covering subject-specific data, historical healthcare interventions, and standardized medical guidelines. The scenario simulation engine 1208 constructs multiple potential next-best actions using a generative AI model 1206 trained on these datasets. The generative AI model 1206 employs architectures such as transformer neural networks or diffusion models to simulate probabilistic outcomes for each potential next-best action, integrating subject-specific parameters, including clinical history and biomolecular markers, to evaluate the effectiveness of these actions.

The scenario simulation engine 1208 generates a probabilistic assessment for each potential next-best action by analyzing simulated trajectories of health outcomes. For example, in a diabetic patient scenario, the scenario simulation engine 1208 may simulate impacts of various insulin dosage adjustments on glycemic control over a defined time horizon. These simulations are evaluated using subject-specific parameters, including historical glycemic variability and response to prior interventions, and stored within the generative AI feedback engine 1204.

The feedback component 1202 is further coupled to a real-time feedback acquisition interface 1210, which serves as the primary conduit for collecting real-time subject data from diverse sources. These sources may include patient monitoring devices (also referred to as patient monitoring device 1218) (e.g., wearable glucose monitors or pulse oximeters), healthcare provider inputs (also referred to as provider input 1220), and quantitative response metrics for previously executed next-best actions (called NBA response metrics 1222). The real-time feedback acquisition interface 1210 is configured to preprocess, encode, and stream collected data to the generative AI feedback engine 1204 for subsequent analysis and refinement. The real-time feedback acquisition interface 1210 implements secure communication protocols, such as FHIR over HTTPS, to ensure data integrity and compliance with healthcare data exchange standards.

An iterative optimization module 1212 is operatively coupled to the scenario simulation engine 1208. The iterative optimization module 1212 may employ reinforcement learning algorithms (also referred to as reinforcement learning algorithm 1214), such as proximal policy optimization (PPO) or deep Q-networks (DQN), to prioritize the potential next-best actions generated by the scenario simulation engine 1208. The prioritization process is guided by predefined metrics, including but not limited to clinical efficacy, patient satisfaction, and healthcare resource utilization. For example, in cases where multiple therapeutic interventions yield comparable clinical outcomes, the optimization module may assign higher priority to actions with reduced resource consumption or higher patient compliance rates.

In some embodiments, the feedback component 1202 may include a pathway refinement system 1216 that may dynamically update the generative AI model 1206 and the scenario simulation engine 1208. The pathway refinement system 1216 may integrate real-time subject data collected by the feedback acquisition interface 1210 and contextual feedback, including deviations in observed outcomes from simulated predictions. For example, if a patient's real-time glucose readings deviate significantly from simulated trajectories following an insulin adjustment, the pathway refinement system 1216 may recalibrate the generative AI model 1206 to improve subsequent simulations. This recalibration process may include retraining or fine-tuning specific layers of the model using updated datasets while preserving the stability of prior learning through techniques such as elastic weight consolidation.

The pathway refinement system 1216 may ensure that the generative AI feedback engine 1204 maintains a high degree of adaptability to dynamically changing subject conditions and environmental factors. For example, when new clinical evidence or treatment guidelines emerge, it may include these changes into the model, thereby ensuring that next-best actions reflect the latest medical standards.

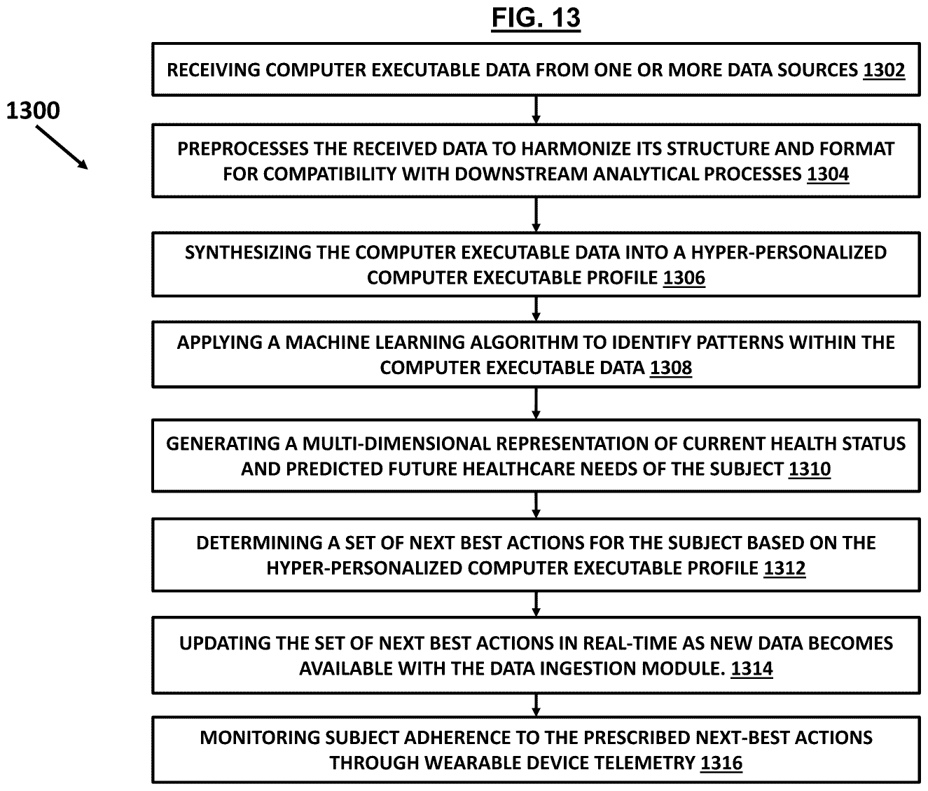

FIG. 13 illustrates a method 1300 for generating the hyper-personalized care pathways or next best actions for the subject, in an embodiment. The method utilizes the SEI system 200 with interrelated components that include such as the data ingestion module, profile generation module 206, pathway generator module 508, and the feedback component 1202 among various other components as discussed in conjunction with various figures. The method 1300 allows integration of the diverse data sources 902, application of advanced machine learning techniques, and dynamic adaptability to real-time updates 1004 for delivering the personalized healthcare interventions or the next best actions.

At step 1302, the method 1300 includes receiving the computer-executable data from the one or more data sources 1302 through the data ingestion module. The data sources 902 may include such as the electronic health records (EHRs) 904, real-time health monitoring devices, laboratory test results (also referred to as laboratory results 908), imaging data 910, patient preferences 912, and socio-economic parameters 914. At step 1304, the data ingestion module preprocesses the received data to harmonize its structure and format for compatibility with downstream analytical processes 1304. For example, unstructured clinical notes from EHRs 904 may undergo natural language processing (NLP), while imaging data 910 is standardized using DICOM protocols.

At step 1306, the method 1300 may include synthesizing, by the profile generation module 206 operatively coupled to the data ingestion module, the computer executable data into a hyper-personalized computer executable profile 1306. The synthesis of the hyper personalized profile may include applying a machine learning algorithm to identify patterns within the computer executable data at 1308. The synthesis of the hyper personalized profile may include generating the multi-dimensional representation of current health status and the predicted future healthcare needs of the subject at 1310.

At step 1312, the pathway generator module 508 may determine the set of next-best actions tailored to the subject's hyper-personalized profile 1312. The next-best actions are dynamically generated computer-traceable interventions optimized for the subject's unique clinical and non-clinical parameters. For example, in a subject with elevated cardiovascular risk, the pathway generator module 508 may prioritize lifestyle modifications, pharmacological therapies, or further diagnostic testing. The pathway generator module 508 may include the predictive analytics engine 936 to quantify the probabilities of potential future medical events, leveraging models such as gradient-boosted decision trees or recurrent neural networks trained on multi-modal datasets. The pathway generator module 508 may generate a prioritized list of recommended interventions based on predefined metrics, including clinical efficacy, patient preferences 912, and socio-economic feasibility.

At step 1314, the method 1300 may include updating the set of next best actions in real-time as new data becomes available with the data ingestion module 1314. The real-time updates 1004 may happen through the data ingestion module. The new data, such as changes in the subject's physiological parameters recorded by wearable devices or feedback from healthcare providers, may be integrated into the hyper-personalized profile. The pathway generator module 508 may dynamically refine the next-best actions by recalibrating the predictive models and prioritization algorithms (also referred to as prioritization algorithm 1020) in response to this real-time data.

In embodiments, the step of synthesizing the computer executable data 916 may include employing the multi-layered neural network 1102 to extract latent variables indicative of clinical and non-clinical parameters. The neural network identifies patterns within the data, such as correlations between lifestyle factors and disease progression, and integrates these insights into the multi-dimensional representation of the subject's current health status 938 and predicted healthcare needs. In embodiments, the SEI system may employ the temporal sequence analytics to model trajectory of the subject's health status based on historical datasets 1024, facilitating predictions of the future medical events.

The method 1300 may be facilitated by the generative AI feedback mechanism (also referred to as generative AI feedback engine 1204) within the feedback component 1202, which may simulate potential next-best actions. The generative AI model 1206 may construct the potential next best actions based on domain-specific datasets, evaluates their effectiveness using subject-specific data, and prioritize them according to predefined success metrics, such as clinical improvement, patient adherence, and resource efficiency. For example, in a diabetic patient, the AI feedback mechanism may simulate various dietary modifications and insulin regimens, selecting the most effective combination for improving glycemic control while considering patient preferences 912.

At 1316, the method 1300 may include monitoring subject adherence to the prescribed next-best actions through wearable device telemetry 1316. In embodiments, data on adherence metrics, such as medication compliance or physical activity levels, may be dynamically fed back into the pathway generator module 508 for recalibration. The adherence monitoring process ensures that the generated care pathways remain actionable and practical for the subject.

The profile generation process may include a data harmonization pipeline to preprocess heterogeneous data formats, enabling seamless integration of the diverse datasets. In embodiments, these techniques include NLP for unstructured text (e.g., clinical notes), Fourier transformations for signal data (e.g., ECG waveforms), and ontology-based mapping for categorical data (e.g., ICD-10 codes). Furthermore, a multi-task deep learning model may be implemented to concurrently identify risk factors, predict disease progression, and generate actionable health insights for ensuring that the synthesized profile is comprehensive and highly predictive.

The hyper-personalized profile may be stored as the multi-dimensional data structure in the non-transitory computer-readable medium 1118. The data structure may integrate demographic parameters 1122, clinical data 1124 encoded in HL7 FHIR schema, and genetic and biomolecular markers 1126 processed through feature extraction algorithms 1104. The stored profile supports interoperability with external healthcare systems via machine-interpretable APIs 1032 for seamless integration with broader healthcare delivery frameworks.

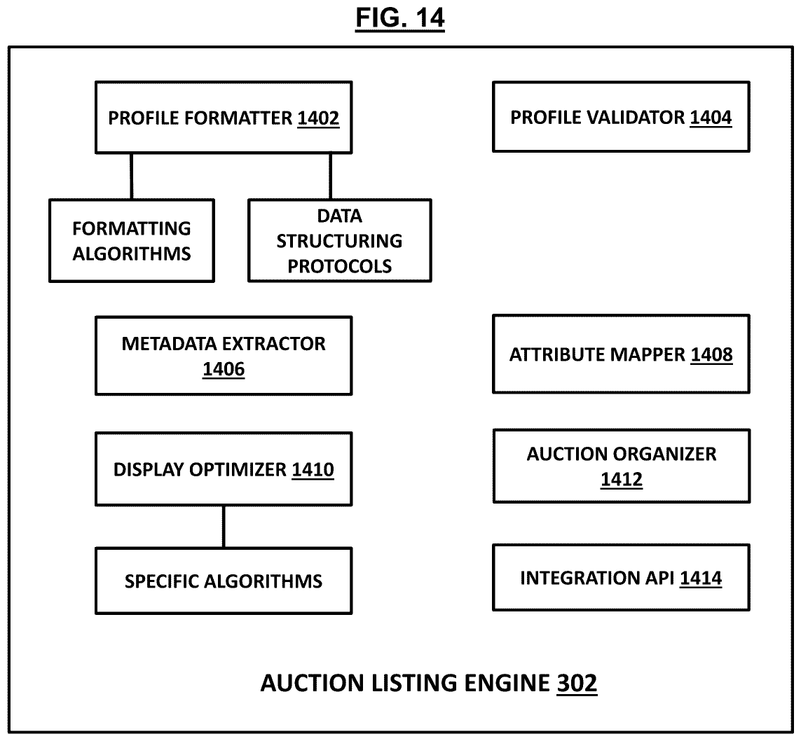

FIG. 14 illustrates the auction listing engine 302 of the reverse auction marketplace platform, in accordance with an embodiment. The auction listing engine 302, as depicted in the FIG. 14, comprises interconnected components including, but not limited to, a profile formatter 1402, a profile validator 1404, a metadata extractor 1406, an attribute mapper 1408, a display optimizer 1410, an auction organizer 1412, and an integration API 1414. In various embodiments, each of these components, functioning as a discrete yet interdependent subsystem, collaboratively provides an accurate, efficient, and regulatory-compliant listing of the hyper-personalized computer executable profiles of various subjects as discussed above within the platform. This facilitates downstream processes such as bid management and dynamic pricing in the reverse auction marketplace.

The profile formatter 1402 may be operatively coupled to the data collection and integration block and the profile generation module. The profile formatter 1402 may be configured to standardize and format the profiles for display within the marketplace platform. The profile formatter 1402 may incorporate formatting algorithms and data structuring protocols to ensure that critical patient information is presented in a standardized manner before listing in the marketplace platform that is compatible with downstream components of the auction listing engine 302. The profile validator 1404 is communicatively linked to the profile formatter 1402 and is responsible for verifying completeness, accuracy, and consistency of patient data, including validation of required attributes, compliance with predefined standards, and resolution of data discrepancies, if any.

The metadata extractor 1406 may be functionally interconnected with the profile validator 1404 and is configured to identify, isolate, and extract key metadata from the profiles. These metadata elements may include, without limitations, demographic information, medical history indicators, and care-specific requirements, which may be critical for enabling subsequent operations of an attribute mapper 1408. The attribute mapper 1408 may be operatively associated with the metadata extractor 1406 and configured to map the extracted metadata to predefined attribute sets utilized by the reverse auction marketplace platform for filtering, categorization, and prioritization of the profiles.

The display optimizer 1410 may interface with an auction organizer 1412 such that visual and logical presentation of the profiles within the marketplace platform adheres to user-centric and performance-optimized guidelines. The display optimizer 1410 may include specific algorithms for visual hierarchy, responsiveness, and clarity, that allows the profiles to be displayed in a manner that improves comprehensibility for healthcare delivery organizations (HDOs) engaging with the marketplace platform. The auction organizer 1412, operationally linked to the display optimizer 1410, manages the logical sequencing, batch processing, and prioritization of profiles for active listing in the auction marketplace. The auction organizer 1412 may further coordinate with the bid management system 304 to facilitate initiation of reverse auction events.

The integration API 1414, positioned as an interface layer, provides a conduit for data interchange between the auction listing engine 302 and external systems, including electronic health record (EHR) systems, regulatory compliance modules, and patient data privacy frameworks. The integration API 1414 ensures that the auction listing engine 302 operates as a cohesive and interoperable entity within the broader SEI system 200 that contains the reverse auction marketplace platform.

In accordance with embodiments, orchestrating of these interrelated subsystems facilitates the auction listing engine 302 to be configured to transform raw data into curated, validated, and dynamically optimized listings that are hyper personalized within the marketplace platform for enabling a transparent, efficient, and patient-centric auction process for healthcare service allocation.

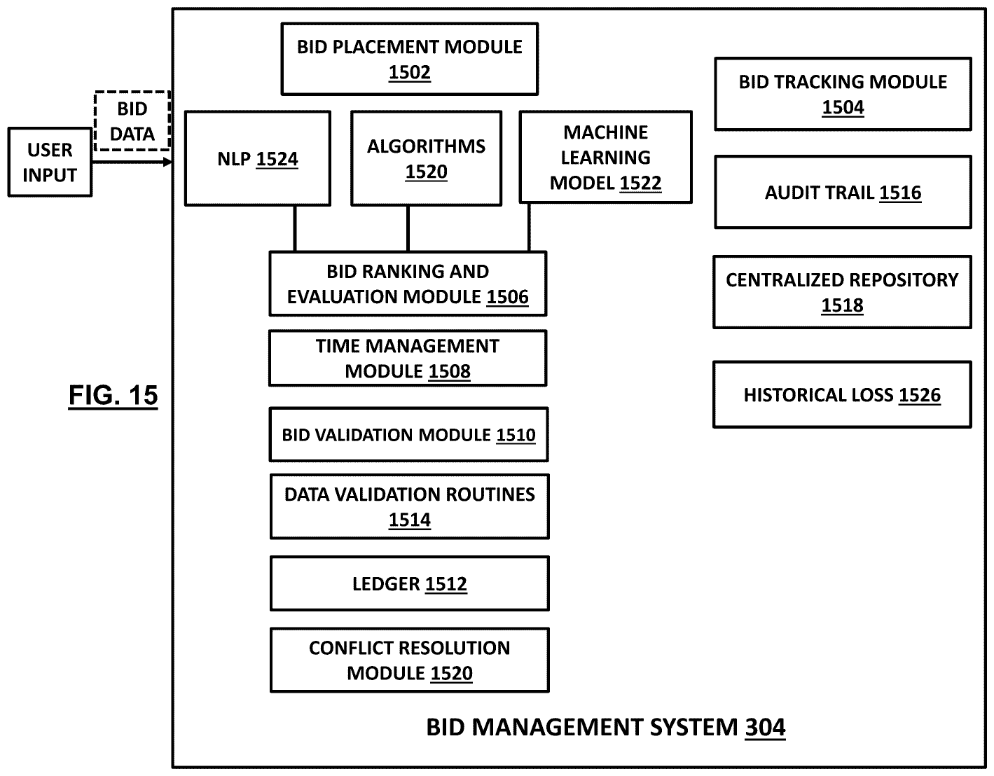

The bid management system 304, as illustrated in the accompanying FIG. 15, is an integral component of the reverse auction marketplace platform, embodying a multifaceted architecture that includes a bid placement module 1502, a bid tracking module 1504, a bid ranking and evaluation module 1506, a time management module 1508. Each of these modules is structurally and functionally interdependent, collectively facilitating a secure, and transparent bidding process for profile acquisition by Healthcare Delivery Organizations (HDOs). The bid placement module 1502 is operatively configured to interface with user input mechanisms, receiving bid data from the HDOs while concurrently validating integrity, authenticity, and compliance of the bid data with predetermined platform standards and regulatory requirements using a bid validation module 1510. After validation, the bid tracking module 1504 maintains a dynamically updated ledger 1512 of bid statuses within an advanced relational database schema. The bid ranking and evaluation module 1506, in conjunction with an external analytics subsystem, may include algorithmic and data-driven methodologies to rank and prioritize bids. In embodiments, the time management module 1508 imposes temporal controls on auction activities. The real time notification system 310 may ensure dissemination of actionable updates to participants through a real-time communication framework.

The bid placement module 1502 may include a computational structure designed to process the bid data received through secure, encrypted communication channels from the HDOs. The bid placement module 1502 and the bid validation module 1510 may be equipped with data validation routines 1514 that examine incoming bid parameters against a predefined schema for compliance with regulatory guidelines such as HIPAA or GDPR and alignment with marketplace-specific bidding constraints. This validation process involves data type verification, boundary checks, and compliance with pricing floor and ceiling thresholds. After successful validation, the bid placement module 1502 may assign a unique cryptographic identifier to each bid, which is subsequently transmitted to the bid tracking module 1504 for further processing. The bid placement module 1502 may be additionally configured to maintain a log of rejected or incomplete bids, providing an audit trail 1516 that can be leveraged for dispute resolution or regulatory audits.

The bid tracking module 1504 includes a centralized repository 1518 for all bid-related data, employing a hierarchical relational database architecture. The bid tracking module 1504 dynamically updates the status of the bids as they transition through various stages of an auction lifecycle, including submission, evaluation, ranking, and closure. The database schema may include indexing and partitioning techniques to enable efficient querying and retrieval of the bid data, thereby supporting high-volume transactional throughput. The bid tracking module 1504 may be further equipped with a conflict resolution mechanism or module 1520 for data consistency in scenarios involving simultaneous bid modifications or cancellations. This utilizes a combination of timestamp-based locking and version control protocols such as through blockchain enabled framework.

The bid ranking and evaluation module 1506 is configured to execute a multi-phase evaluation process. The bid ranking and evaluation module 1506 initially applies algorithms 1520 such as rule-based heuristic algorithms to filter bids based on static parameters such as bid amount, HDO accreditation, and resource availability. Subsequently, an advanced machine learning model 1522 may analyze historical bidding data, provider performance metrics, and patient outcomes to generate a weighted ranking of the bids. The bid ranking and evaluation module 1506 may incorporate natural language processing (NLP) 1524 capabilities to interpret textual data, such as provider reviews or patient feedback, thereby enriching the bid evaluation process. In embodiments, results of this evaluation may be stored within a dedicated cache for rapid access by the auction listing engine.

The time management module 1508 imposes strict temporal constraints on bidding activities for adherence to auction schedules and deadlines. The time management module 1508 may generates auction timelines based on configurable parameters, such as patient profile priority levels and HDO engagement metrics. The time management module 1508 monitors countdowns to auction closures and triggers automated actions, such as bid retractions or deadline extensions, in response to predefined conditions. The time management module 1508 may maintain a historical log (also referred to as historical logs) 1526 of auction timings, which can be utilized for performance analysis and optimization.

The bid management system 304 may be coupled with the real time notification system 310. The real time notification system 310 may utilize a push-based architecture to disseminate updates to platform participants. The real time notification system 310 may support multi-channel delivery mechanisms, including email, SMS, and in-app alerts, ensuring comprehensive reach and engagement. Notifications may include a wide array of events, such as bid confirmations, auction status changes, and bidding results. The real time notification system may integrate with the bid tracking module 1504 and the time management module 1508 for timely and contextually relevant message delivery. The real time notification system may include message queuing and prioritization techniques to handle high notification volumes without compromising latency.

In various embodiments, the bid management system 304 may be architected to operate within a broader framework of the reverse auction marketplace platform. The modular design facilitates scalability, enabling the SEI system 200 to accommodate varying participant loads and auction complexities.

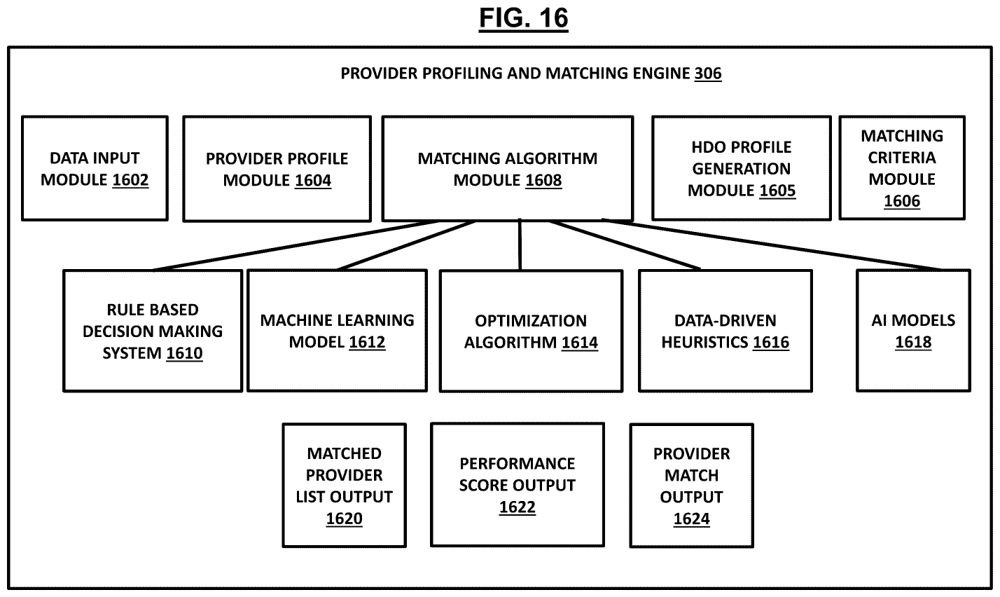

The provider profiling and matching engine 306, as depicted in the FIG. 16, includes a plurality of interconnected subsystems, each of which may be functionally interdependent and jointly facilitating an efficient, personalized, and dynamic matching of the healthcare providers to patient-specific requirements within the reverse auction marketplace or healthcare delivery environment according the information contained in the computer executable profiles. These subsystems, including but not limited to, a data input module 1602, a provider profile module 1604, a matching criteria module 1606, a matching algorithm module 1608, a matched providers list output 1620, a performance score output 1622, and a provider match output 1624, collaboratively operate to ensure an accurate and optimal selection of the healthcare providers based on a combination of provider attributes and patient needs.

The data input module 1602 may be operably connected to multiple data sources, including but not limited to internal databases, third-party healthcare systems, and electronic health records (EHR), and serves as a foundational entry point for an acquisition and initial processing of raw data regarding the healthcare providers. This raw data may include diverse provider-related attributes, such as professional qualifications, specialization, operational capacity, historical performance data, geographical location, and other relevant operational metrics that are each in computer executable format. The data input module 1602 may be configured to receive dynamic data updates, including real-time scheduling information, resource availability, and performance metrics, ensuring the provision of up-to-date information for accurate profile creation.

The provider profile module 1604, which is communicatively linked to the data input module 1602, may be configured as an intermediary in the transformation of the raw input data into standardized, structured, and coherent computer executable provider profiles. An HDO profile generation module 1605, situated in close association with the provider profile module 1604, synthesizes various pieces of the raw data into a comprehensive provider profile, comprising data points that represent a healthcare provider's expertise, capacity, historical performance, geographic reach, and other relevant characteristics. The HDO profile generation module 1605 ensures that the generated provider profiles are in a format that adheres to the standards required by downstream components and are compatible with the matching process. The generated profile provides a key dataset upon which the matching algorithm operates.

The matching criteria module 1606, operatively associated with the HDO profile generation module 1605, is configured to receive and incorporate user-specific requirements for the matching process. The matching criteria module 1606 defines the set of parameters by which a healthcare provider's profile will be evaluated and compared to the needs of patients, healthcare delivery organizations (HDOs), or other healthcare stakeholders. These criteria may include clinical attributes such as required specialties, treatment modalities, or patient volume; operational factors like provider availability, location, and capacity; or performance-related data such as provider success rates, patient feedback, and prior outcomes. The matching criteria module 1606 may further include configurable rules and filters, allowing customization based on specific patient or organizational requirements, so that the resulting matches are tailored to the unique context of a requestor.

The matching algorithm module 1608, functionally interconnected with both the provider profile module 1604 and the matching criteria module 1606, is configured to execute and apply sophisticated algorithmic techniques to match providers to patients or HDOs based on the inputted profiles and criteria. The matching algorithm module 1608 may utilize a variety of methodologies, including a rule-based decision-making system 1610, a machine learning model 1612, an optimization algorithm 1614, and a data-driven heuristics 1616, to determine suitability of providers. In certain embodiments, the matching algorithm module 1608 may incorporate artificial intelligence (AI) models 1618 that leverage historical provider performance data, patient outcomes, and other predictive analytics to generate more precise and effective matches. The matching algorithm module 1608 may further utilize real-time data feeds, including availability information and dynamic scheduling, to ensure that the matching process reflects the most current and actionable provider attributes.

The matched providers list output 1620, generated as a result of the operation of the matching algorithm module 1608, provides a prioritized list of healthcare providers who are deemed the best matches based on the specified criteria and provider profiles. The list may be structured according to various relevance metrics, such as proximity, clinical competency, availability, or performance scores. The matched providers list output 1620 serves as a foundation for the HDOs or patients to review the top candidates for further engagement, providing them with a curated set of options that have been dynamically optimized for their specific needs.

The performance score output 1622 is configured to offer a quantitative or qualitative assessment of each matched provider based on a variety of performance metrics. These metrics may include historical patient outcomes, treatment success rates, patient satisfaction surveys, and operational efficiency indicators. The performance score output 1622 may function as an additional layer of evaluation, enabling the HDOs and patients to not only assess providers based on their immediate match to the criteria but also to consider the provider's track record and reputation within the broader healthcare community. The performance score (also referred to as performance score output 1622) can therefore act as a decision-making aid that helps in the matching process by incorporating a more holistic view of provider performance.

The provider match output 1624 represents a final output of the entire profiling and matching process. This is presented to the relevant stakeholders, such as healthcare providers, HDOs, or patients, and acts as a recommendation or invitation for further interaction, such as scheduling an appointment or initiating a referral. The provider match output 1624 is based on a combination of the matched providers list (also referred to as matched providers list output 1620) and the performance score 1622, thereby providing a comprehensive and multi-dimensional view of the best provider options available. Additionally, this output may be associated with various follow-up actions, such as scheduling mechanisms, resource allocation processes, or contract negotiations, depending on the context of the reverse auction marketplace.

In accordance with various embodiments, the provider profiling and matching engine 306 operates as an integrated, end-to-end system that facilitates matching of the healthcare providers with patient or organizational needs. The marketplace platform ensures that raw data is transformed into curated, validated, and dynamically optimized provider profiles that align with user-specific criteria. Furthermore, the matching algorithms employed by the marketplace platform ensure that the matches are not only accurate but also contextually relevant, taking into account a multitude of dynamic factors such as real-time availability, resource constraints, and historical performance data. The integration of the performance score output 1622 adds an additional layer of transparency and decision-making support, thereby improving the overall matching process and ensuring that healthcare delivery organizations and patients are presented with the most suitable provider options available.

The modular and scalable nature of the provider profiling and matching engine 306 allows it to be adapted to a variety of use cases within the healthcare sector, whether it be for one-to-one patient-provider matching, large-scale provider sourcing for healthcare organizations, or dynamic matchmaking within a reverse auction framework. This flexibility, coupled with the system's ability to process and integrate vast amounts of data, positions the provider profiling and matching engine 306 as a key enabling technology within modern healthcare delivery ecosystems.

The various components described herein and/or illustrated in the figures may be embodied as hardware-enabled modules and may be a plurality of overlapping or independent electronic circuits, devices, and discrete elements packaged onto a circuit board to provide data and signal processing functionality within a computer. An example might be a comparator, inverter, or flip-flop, which could include a plurality of transistors and other supporting devices and circuit elements. The modules that include electronic circuits process computer logic instructions capable of providing digital and/or analog signals for performing various functions as described herein. The various functions can further be embodied and physically saved as any of data structures, data paths, data objects, data object models, object files, database components. For example, the data objects could include a digital packet of structured data. Example data structures may include any of an array, tuple, map, union, variant, set, graph, tree, node, and an object, which may be stored and retrieved by computer memory and may be managed by processors, compilers, and other computer hardware components. The data paths can be part of a computer CPU that performs operations and calculations as instructed by the computer logic instructions. The data paths could include digital electronic circuits, multipliers, registers, and buses capable of performing data processing operations and arithmetic operations (e.g., Add, Subtract, etc.), bitwise logical operations (AND, OR, XOR, etc.), bit shift operations (e.g., arithmetic, logical, rotate, etc.), complex operations (e.g., using single clock calculations, sequential calculations, iterative calculations, etc.). The data objects may be physical locations in computer memory and can be a variable, a data structure, or a function. Some examples of the modules include relational databases (e.g., such as Oracle® relational databases), and the data objects can be a table or column, for example. Other examples include specialized objects, distributed objects, object-oriented programming objects, and semantic web objects. The data object models can be an application programming interface for creating HyperText Markup Language (HTML) and Extensible Markup Language (XML) electronic documents. The models can be any of a tree, graph, container, list, map, queue, set, stack, and variations thereof, according to some examples. The data object files can be created by compilers and assemblers and contain generated binary code and data for a source file. The database components can include any of tables, indexes, views, stored procedures, and triggers.

In an example, the embodiments herein can provide a computer program product configured to include a pre-configured set of instructions, which when performed, can result in actions as stated in conjunction with various figures herein. In an example, the pre-configured set of instructions can be stored on a tangible non-transitory computer readable medium. In an example, the tangible non-transitory computer readable medium can be configured to include the set of instructions, which when performed by a device, can cause the device to perform acts similar to the ones described here.

The embodiments herein may also include tangible and/or non-transitory computer-readable storage media for carrying or having computer-executable instructions or data structures stored thereon. Such non-transitory computer readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a special purpose computer or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network. If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

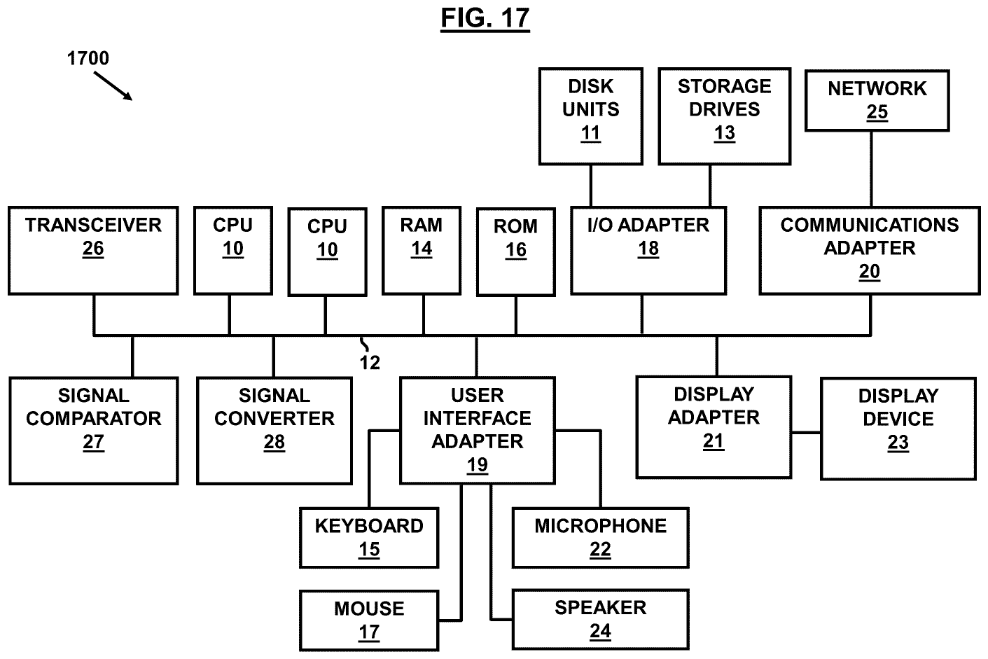

A representative hardware environment for practicing the embodiments herein is depicted in FIG. 17, with reference to FIGS. 1 through 16. This schematic drawing illustrates a hardware configuration of an information handling/computer system 1700 in accordance with the embodiments herein. The system 1700 comprises at least one processor or central processing unit (CPU) 10. The CPUs 10 are interconnected via system bus 12 to various devices such as a random-access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system 600 can read the inventive instructions on the program storage devices and follow these instructions to execute the methodology of the embodiments herein. The system 600 further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network, and a display adapter 21 connects the bus 12 to a display device 23 which may be embodied as an output device such as a monitor, printer, or transmitter, for example. Further, a transceiver 26, a signal comparator 27, and a signal converter 28 may be connected with the bus 12 for processing, transmission, receipt, comparison, and conversion of electric or electronic signals.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Those skilled in the art will recognize that the embodiments herein can be practiced with modification within the spirit and scope of the appended claims.

What is claimed is:

1. A system for generating hyper-personalized care pathways for a subject, the system comprising:
a data ingestion module configured to:
receive and process computer executable data from one or more data sources through encrypted communication channels,
wherein the computer executable data includes at least one of electronic health records (EHRs), real-time health monitoring data, laboratory results, imaging data, patient preferences, and socio-economic parameters;
implement blockchain-based verification protocols to ensure data integrity during transmission;
a profile generation module operatively coupled to the data ingestion module, wherein the profile generation module is configured to:
generate encrypted data containers for storing the computer executable data;
synthesize the computer executable data into a hyper-personalized computer executable profile by applying a machine learning algorithm and predictive analytics while maintaining HIPAA-compliant access controls;
generate a multi-dimensional computer executable representation of current health status and predicted future healthcare needs of the subject within the encrypted data containers; and
a pathway generator module operatively coupled to the profile generation module, wherein the pathway generator module is configured to:
determine a set of next best actions for the subject based on the hyper-personalized computer executable profile while maintaining data privacy through role-based access controls;
transmit the next best actions through secure communication protocols, the next best actions comprising dynamically generated computer traceable interventions tailored to one or more clinical and non-clinical parameters associated with the subject; and
update the set of next best actions in real-time as new data becomes available with the data ingestion module while maintaining an encrypted audit trail of all updates.

2. The system of claim 1, wherein the profile generation module further comprises:
a multi-layered neural network configured to analyze synthesized computer executable data, wherein the neural network employs a feature extraction technique to identify one or more latent variables indicative of the clinical and non-clinical parameters of the subject; and
a predictive modeling engine incorporating temporal sequence analytics to anticipate a change in the current health status of the subject, the predictive modeling engine being trained on a plurality of historical datasets associated with the subject to refine the hyper-personalized computer executable profile iteratively.

3. The system of claim 1, wherein the hyper-personalized computer executable profile comprises:
a multidimensional data structure stored in a non-transitory computer-readable medium, wherein the data structure integrates one or more of:
demographic parameters mapped to healthcare utilization models;
clinical data encoded in accordance with HL7 FHIR schema for standardization and interoperability; and
genetic and biomolecular markers processed through feature extraction algorithms.

4. The system of claim 3, wherein the hyper-personalized computer executable profile further comprises a set of predictive analytics data, wherein the set of predictive analytics data includes a probabilistic risk assessment, quantified using gradient-boosted decision trees trained on a multi-modal dataset to assess probabilities of one or more future medical events.

5. The system of claim 3, wherein the hyper-personalized computer executable profile further comprises a dynamic feedback data set, wherein the data set reflects adjustments to care pathways based on real-time subject monitoring data and previous intervention outcome.

6. The system of claim 3, wherein the hyper-personalized computer executable profile further comprises a structured data interface, wherein the structured data interface is configured to facilitate extraction of actionable healthcare insights from the data structure and allows integration with an external healthcare delivery system using machine-interpretable APIs.

7. The system of claim 1, further comprising:
a security module configured to:
implement HIPAA-compliant encryption protocols for all stored and transmitted data;
maintain blockchain-based verification of data integrity;
generate secure audit logs of all data access and modifications; and
enforce role-based access controls for all system interactions.

8. The system of claim 1, further comprising a feedback component that is configured to incorporate outcomes of the next best actions into the hyper-personalized computer executable profile to refine subsequent recommendations by the pathway generator module, wherein the feedback component includes a generative AI feedback engine, the generative AI feedback engine comprises a scenario simulation engine configured to construct multiple potential next best actions by leveraging a generative AI model trained on domain-specific computer executable datasets including subject data, wherein the model generates probabilistic outcomes and evaluates effectiveness of the potential next best actions based on subject-specific data.

9. The system of claim 8, wherein the AI feedback component is coupled to a real-time feedback acquisition interface, wherein the feedback acquisition interface is configured to collect the subject data from one or more of a patient monitoring device, healthcare provider input, and next best action response metrics to refine simulated next best actions, and wherein the AI feedback component further comprising:
an iterative optimization module operatively coupled to the scenario simulation engine, wherein the scenario simulation engine employs a reinforcement learning algorithm to prioritize the potential next best actions based on predefined metrics, including clinical efficacy, patient satisfaction, and resource utilization; and
a pathway refinement system configured to dynamically update the potential next best actions by incorporating real-time changes in the subject data and contextual feedback into the generative AI model.

10. The system of claim 1, wherein the profile generation module implements:
encrypted data containers for storing patient profiles;
secure multi-party computation protocols for distributed data processing;
privacy-preserving machine learning algorithms that maintain data confidentiality during analysis; and
secure key management protocols for controlling access to encrypted data.

11. The system of claim 1, wherein the pathway generator module further comprises a next-best-actions generator configured to dynamically analyze the hyper-personalized profile of the subject synthesized by the profile generation module in conjunction with a plurality of computer executable clinical guidelines, historical treatment efficacy datasets, and real-world evidence databases, wherein the next-best-actions generator utilizes a machine learning algorithm, including supervised learning models and reinforcement learning frameworks, to generate the set of next best actions, and wherein the next-best-actions generator is operatively coupled to a predictive modeling engine that applies predictive modeling techniques to account for temporal factors and treatment timelines by generating intervention schedules optimized based on specified parameters.

12. The system of claim 1, wherein the pathway generator module further comprises a multi-tiered prioritization engine configured to stratify the set of next-best-actions into categories comprising immediate, short-term, and long-term interventions, wherein the multi-tiered prioritization engine incorporates a prioritization algorithm to evaluate one or more specified computer executable parameters, and wherein a prioritization outcome is communicated to provider system or a subject system through a user interface block in real time.

13. A method for generating hyper-personalized care pathways for a subject, comprising:
receiving, by a data ingestion module, computer executable data from one or more data sources, wherein the computer executable data includes at least one of electronic health records (EHRs), real-time health monitoring data, laboratory results, imaging data, patient preferences, and socio-economic parameters;
synthesizing, by a profile generation module operatively coupled to the data ingestion module, the computer executable data into a hyper-personalized computer executable profile, wherein the synthesis includes:
applying a machine learning algorithm to identify patterns within the computer executable data; and
generating a multi-dimensional representation of current health status and predicted future healthcare needs of the subject;
determining, by a pathway generator module operatively coupled to the profile generation module, a set of next best actions for the subject based on the hyper-personalized computer executable profile, wherein the next best actions comprise dynamically generated computer traceable interventions tailored to one or more clinical and non-clinical parameters associated with the subject; and
updating the set of next best actions in real-time as new data becomes available with the data ingestion module.

14. The method of claim 13, wherein synthesizing the computer executable data further comprises:
employing a multi-layered neural network to extract one or more latent variables from the data, wherein the latent variables are indicative of clinical and non-clinical parameters of the subject; and
applying temporal sequence analytics to predict a change in the subject's health status based on historical datasets.

15. The method of claim 13, wherein determining the set of next best actions further comprises:
analyzing the hyper-personalized computer executable profile using a predictive analytics engine to quantify the probabilities of one or more future medical events; and
generating a prioritized list of interventions based on predefined metrics, including clinical efficacy, subject preferences, and socio-economic feasibility.

16. The method of claim 13, further comprising:
storing the hyper-personalized computer executable profile as a multi-dimensional data structure in a non-transitory computer-readable medium, wherein the data structure integrates one or more of:
demographic parameters mapped to healthcare utilization models;
clinical data encoded in accordance with HL7 FHIR schema; and
genetic and biomolecular markers processed through feature extraction algorithms.

17. The method of claim 13, wherein updating the set of next best actions further comprises incorporating real-time feedback received from patient monitoring devices, healthcare provider inputs, and outcome metrics of previously implemented actions into the pathway generator module to dynamically refine subsequent next based actions.

18. The method of claim 17, further comprising:
utilizing a generative AI feedback mechanism to simulate one or more potential next best actions by constructing the potential next best actions using a generative AI model trained on domain-specific datasets; and
evaluating effectiveness of the one or more potential next best actions based on subject-specific data and predefined success metrics.

19. The method of claim 13, further comprising monitoring subject adherence to the next best actions through an integration of wearable device telemetry, wherein adherence metrics are dynamically fed back into the pathway generator module to recalibrate the next best actions.

20. The method of claim 13, wherein synthesizing the computer executable data further comprises:
preprocessing heterogeneous data formats using a data harmonization pipeline that includes one or more of natural language processing for unstructured text, Fourier transformations for signal data, and ontology-based mapping for categorical data; and
implementing a multi-task deep learning model to concurrently perform of or more of identifying risk factors, predicting disease progression, and generating health insights.
